SEQUENCE LISTING (10 sequences)

SEQ ID NO: 1 - DNA, 18 bp, Artificial Sequence
  a-SMA forward primer
caaagccggccttacaga

SEQ ID NO: 2 - DNA, 17 bp, Artificial Sequence
  a-SMA reverse primer
aggccagccaagcactg

SEQ ID NO: 3 - DNA, 18 bp, Artificial Sequence
  ERG forward primer
ggagtgggcggtgaaaga

SEQ ID NO: 4 - DNA, 20 bp, Artificial Sequence
  ERG reverse primer
aaggatgtcggcgttgtagc

SEQ ID NO: 5 - DNA, 23 bp, Artificial Sequence
  GAPDH forward primer
caaggtcatccatgacaactttg

SEQ ID NO: 6 - DNA, 20 bp, Artificial Sequence
  GAPDH reverse primer
gggccatccacagtcttctg

SEQ ID NO: 7 - DNA, 20 bp, Artificial Sequence
  SMC forward primer
ctggctgcagcttattgatg

SEQ ID NO: 8 - DNA, 20 bp, Artificial Sequence
  SMC reverse primer
ctgagagagtggatcgaggg

SEQ ID NO: 9 - DNA, 22 bp, Artificial Sequence
  TGFb2 forward primer
ggtaccttgatgccatcccgcc

SEQ ID NO: 10 - DNA, 24 bp, Artificial Sequence
  TGFb2 reverse primer
gcactctggcttttgggttctgca

(19) United States
(12) Patent Application Publication
(10) Pub. No.: US 2023/0174948 A1
(43) Pub. Date: Jun. 8, 2023

(54) METHOD OF GENERATING ENDOTHELIAL CELLS

(71) Applicant: IMPERIAL COLLEGE INOVATIONS LIMITED, London (GB)

(72) Inventors: Gabor Foldes, London (GB); Qasim A. Majid, London (GB); Sian E. Harding, London (GB)

(21) Appl. No.: 17/905,691

(22) PCT Filed: Mar. 5, 2021
(86) PCT No.: PCT/GB2021/050558

§ 371 Date: Sep. 6, 2022

(30) Foreign Application Priority Data
Mar. 6, 2020 (GB) 2003309.8

(51) Int. Cl. C12N 5/071; A61K 35/44; A61P 9/00

(52) U.S. Cl. CPC C12N 5/0691; A61K 35/44; A61P 9/00; C12N 2501/16; C12N 2501/105; C12N 2501/115; C12N 2501/155; C12N 2501/165; C12N 2506/02; C12N 2506/45; C12N 2513/00; C12N 2533/54

(57) ABSTRACT

The present invention relates to endothelial cells and methods of generating endothelial cells from pluripotent stem cells.

### FIELD OF THE INVENTION

The present invention relates to endothelial cells and methods of generating endothelial cells from pluripotent stem cells.

### BACKGROUND OF THE INVENTION

Primary endothelial cells are a promising cell source for therapeutic applications as well as for modelling angiogenesis and cardiovascular function and disease in vitro. These cells can be derived and expanded from circulating blood or from solid tissues (such as those derived from the endothelium of umbilical cord blood veins, human aortic endothelial cells and human lung endothelial cells). Despite this, scarce availability of donor tissue, low expansion rate and loss of differentiated phenotype in culture, largely limit their usage. Moreover, there is considerable heterogeneity in primary endothelial cells derived from different tissue sources, reflecting the differences between endothelial populations from arteries and veins, and even between small or large vessels and normal or cancerous vessels. All these factors must, therefore, be taken into consideration when choosing a cell source for in vitro and in vivo use.

Human pluripotent stem cells (hPSC), which include human embryonic stem cells (hESC) and human induced pluripotent stem cells (hiPSC), represent a very promising alternative cell source, as they can self-renew indefinitely in culture while maintaining the ability to become any cell type in the human body. Various protocols have been developed to differentiate the hPSCs in vitro into pluripotent stem cell-derived endothelial cells (hPSC-ECs). These cells express endothelial markers, grow as a homogenous cell monolayer with cobblestone-morphology, show proliferative potential, and can form vessel-like networks in vitro and in vivo when supported by a matrix. However, conventional two-dimensional culture approaches limit the number of cells which may be produced in these methods. These conventional methods also rely on the use of non-GMP (non-human animal derived components) such as Matrigel™ which limits their use for therapeutic applications.

### SUMMARY OF THE INVENTION

The present inventors have developed a new 3-D culture methodology using stirred-tank bioreactors as a scalable platform for the production hPSC-derived endothelial cells. This method allows for the large-scale production of endothelial cells, increasing their therapeutic potential, as clinical application requires millions of cells for transplantation. In addition, the inventors have found that conventional 2D-culture methods do not produce stable endothelial cell lines, with the cellular phenotype drifting to non-endothelial cell lineages within a matter of days or within one to two passages of the cells. In contrast, the 3-D culture methodology developed by the present inventors produces endothelial cells which maintain their phenotype for weeks.

Accordingly the invention provides a method of generating endothelial cells from pluripotent stem cells, wherein said method comprises: (a) culturing the pluripotent stem cells in a 3-D suspension culture; and (b) inducing the pluripotent stem cells to undergo endothelial differentiation, wherein inducing comprises: (i) culturing the pluripotent stem cells for about 24 hours in a first endothelial differentiation medium comprising Activin-A, BMP-4, FGF-2 and VEGF; and (ii) replacing the first endothelial differentiation medium with a second endothelial differentiation medium comprising BMP-4, FGF-2 and VEGF; wherein the pluripotent stem cells form cellular aggregates in the suspension.

The pluripotent stem cells employed in methods of the invention may be human pluripotent stem cells, optionally human induced pluripotent stem cells or human embryonic stem cells. The human pluripotent stem cells may be H7 cells, IMR 90-4 cells, RC11 cells and/or HUES7 cells, preferably wherein the human pluripotent stem cells are H7 or IMR 90-4 cells.

In accordance with the invention, said endothelial cells may be: (a) CD31⁺ and/or NRP-1^{lo}, preferably CD31⁺ and NRP-1^{lo}; and/or (b) human endothelial cells. Said endothelial cells may be KDR⁻ or KDR^{lo}.

In accordance with the invention said endothelial cells may be human endothelial cells which express at least one gene selected from: an F-subgroup Sox transcription factor, preferably SOX7, SOX17 and/or SOX18; LYL1; YAP1; HCLs1; HOXB3; HOXB7; ZNF300; CYP1B1; LYL1; YAP1; HCLs1; HOXB3; HOXB7; ZNF300; CYP1B1; VEGF-A; VE-Cadherin; PNP; OGDH; NOTCH1; NOTCH2; GLB1; ETV2; Ephrin B2; COL1A1; COL3A1; CD31; APLNR; PLAU; MMP9; ACVR1B; HGF; ERG; Tie2; Angiotensin II; ICAM2; VWF; Fli-1; ALK1; SMAD7; FSP1 and/or SMA-αβ.; wherein preferably; (a) the expression of at least one of SOX7, SOX17, SOX18, LYL1, YAP1, HOXB7, CYP1B1, VEGF-A, VE-Cadherin, PNP, OGDH, NOTCH1, NOTCH2, GLB1, ETV2, Ephrin B2, COL1A1, COL3A1, CD31, and/or APLNR is expressed at higher levels increased compared with the expression level in endothelial cells generated using traditional 2-D methods and/or native endothelial cells; and/or (b) the expression level of at least one of HGF and/or FSP1 is expressed at lower levels decreased compared with the expression level in endothelial cells generated using traditional 2-D methods and/or native endothelial cells.

In accordance with the invention said endothelial cells may additionally express one or more of VE-Cadherin, ETS-related gene (ERG), Tie2, Angiotensin II, ICAM2, VWF, , ETV-2, Fli-1, ALK1, SMAD7, APLNR, and/or SMA-αβ2 SOX7, SOX17, SOX18, LYL1, YAP1, HCLsl, HOXB3, HOXB7, ZNF300, CYP1B1, VEGF-A, VE-Cadherin, PNP, OGDH, NOTCH1, NOTCH2, GLB1, ETV2, Ephrin B2, COL1A1, COL3A1, CD31, APLNR, PLAU, MMP9, ACVR1B, HGF, ERG, Tie2, Angiotensin II, ICAM2, VWF, Fli-1, ALK1, SMAD7, FSP1, SMA-αβ, EGF, NRG1, FGF4, CXCL16, IL8, FGF1, FGF7, LEP, VEGFC, TIMP4, ADAMTS1, PF4, CSF2, ANG, PROK1, PLG, CCL2, GDNF, PDGFB, TGFB1, PRL, FGF2, VASH1, IL1B, PDGFA, MMP8, TYMP, PIGF, THBS2, PSPN, SERPINB5, CCL3, ANGPT1, SERPINF1, HBEGF, PTX3, TIMP1, ARTN, IGFBP3, IGFBP1, AREG, COL18A1, EDN1, DPP4, F3, IGFBP2, THBS1, ENG, ANGPT2, SERPINE1.

In accordance with the methods of the invention: (a) the concentration of each of Activin-A, BMP-4, FGF-2 and VEGF in the first endothelial differentiation medium may be independently selected from a concentration in the range of about 1 ng/ml to 100 ng/ml; wherein preferably the concentration of Activin-A, BMP-4 and FGF-2 is independently selected from a concentration in the range of about 5 to 25 ng/ml and VEGF is present in a concentration in the range of about 5 to 50 ng/ml; more preferably wherein each of Activin-A, BMP-4, FGF-2 and VEGF is present at a concentration of about 10 ng/ml; and/or (b) the concentration of each of BMP-4, FGF-2 and VEGF in the second endothelial differentiation medium may be independently selected from a concentration in the range of 1 ng/ml to 100 ng/ml; wherein preferably the concentration of BMP-4 and FGF-2 is independently selected from a concentration in the range of about 5 to 25 ng/ml and VEGF is present in a concentration in the range of about 0 to 50 ng/ml; more preferably wherein each of BMP-4, FGF-2 and VEGF is present at a concentration of about 10 ng/ml.

Said endothelial cells may exhibit a stable endothelial cell phenotype.

Said endothelial cells may exhibit a stable endothelial cell phenotype for at least three to 12 passages, preferably five to ten passages.

Said endothelial cells (preferably human endothelial cells) may be capable of vascular structure formation in vivo.

Said endothelial cells may be functional without co-culture, preferably said endothelial cells produce 3-D vascular and/or tubular structures in vitro without co-culture.

Said endothelial cells may exhibit a microvascular endothelial cell phenotype, preferably a cardiac microvascular endothelial cell phenotype.

In accordance with the invention, said 3-D suspension culture of the pluripotent stem cells may be carried out in a stirred-tank bioreactor. Said stirred-tank bioreactor may be operated at between about 20 to 100 rpm, preferably at about 50 rpm.

The cultures of the invention may be: (a) serum-free; (b) free of non-human serum albumin; (c) free from non-human animal derived components; and/or (d) carried out using human serum albumin as the only animal derived protein in the culture medium.

In accordance with the methods of the invention the first endothelial differentiation medium may be mTESR1 medium and the second endothelial differentiation may be StemLine medium.

In accordance with the methods of the invention, the culture vessel used for the 3-D suspension culture may be feeder-cell free and/or coating-free, preferably both feeder-cell free and coating free.

In accordance with the methods of the invention an adaptive feed rate may be used to reduce the concentration of lactate within the 3-D suspension culture.

Said pluripotent stem cells may be expanded prior to 3-D suspension culture, preferably wherein said expansion comprises culturing the pluripotent stem cells in a pluripotent stem cell expansion medium and passaging the pluripotent stem cells when confluency of at least about 70% is achieved. Pluripotent stem cell aggregates may be retained when transferring the expanded pluripotent stem cells to the 3-D suspension culture.

Methods of the invention may further comprise the step of isolating one or more endothelial cell, wherein preferably the step of isolating one or more endothelial cell comprises: (a) Fluorescence-activated cell sorting (FACS), immunoprecipitation and/or Magnetic-activated cell sorting (MACS); or (b) culturing a differentiated cell culture obtained by the method of any one of the preceding claims with a cell culture medium comprising a carbohydrate exclusively metabolised by human endothelial cells. Preferably said method comprises only one step of isolating one or more endothelial cell.

The present invention also provides a method of isolating an endothelial cell from a differentiated cell culture, comprising culturing a differentiated cell culture with a cell culture medium comprising a carbohydrate exclusively metabolised by endothelial cells.

The present invention also provides a method of maintaining an endothelial cell phenotype comprising culturing one or more endothelial cell with a cell culture medium comprising a carbohydrate exclusively metabolised by endothelial cells.

In accordance with methods of the invention, the cell culture medium may be glucose-free, preferably wherein the carbohydrate is selected from one or more of lactose, meso-tartaric acid, dextrin, maltotriose, D-turanose inosine and/or alpha-keto-glutaric acid.

Said endothelial cell phenotype may comprise expression of CD31⁺ and NRP-1^{lo}.

Said endothelial cell phenotype is maintained for at least about three to 12 passages, preferably five to ten passages. The cell culture medium may comprise: (a) VEGF, optionally at a concentration of from about 1 to 100 ng/ml, preferably about 50 ng/ml; (b) a STAT3 inhibitor; and/or (c) heparin, optionally at a concentration of from about 1 to 250 mg/ml, preferably about 100 mg/ml.

The invention also provides an endothelial cell, preferably a human endothelial cell, obtainable by any one of the methods of the invention.

The invention also provides a composition comprising an endothelial cell, preferably a human endothelial cell obtainable by any one of the methods of the invention, and optionally a pharmaceutically acceptable excipient.

The invention also provides a human endothelial cell according to the invention, or a composition according to the invention for use in the treatment of cardiovascular disease.

The invention also provides a use of a human endothelial cell according to the invention, or a composition according to the invention, in the manufacture of a medicament for the treatment of vascular disease.

### BRIEF DESCRIPTION OF THE DRAWINGS

FIG. 1: Comparison of 2-D and 3-D differentiation protocols: (A) controls and strategy used for FACS/gating set up and auto-fluorescence exclusion (FACS gates; Q1: CD31/NRP-1 -/+, Q2: CD31/NRP-1 +/+, Q3: CD31/NRP-1 +/-, Q4: CD31/NRP-1 -/- cells). (B) validation of endothelial enrichment by flow cytometry. (C) CD31+ endothelial cells. (D) decreased NRP-1 expression in 3-D protocol as compared with 2-D differentiation; Mann-Whitney, *P<0.05, n=17 for 2D and n=7 for 3-D independent isolations, respectively. (E) distribution of single- and double-positive endothelial cells during sorting.

FIG. 2: Flow cytometry assessment of forward scatter parameter of cells differentiated using 2-D and 3-D differentiation protocols. (A) FSC is a measurement of the amount of the laser beam that passes around the cell, i.e. a relative size for the cell. (B) Standard deviation of FSC (rSD) shows homogeneity of endothelial cell size. Mann-Whitney, *P<0.05, n=17 for 2D and n=7 for 3D independent isolations, respectively.

FIG. 3: Human PSC-derived endothelial cells differentiated using 2-D protocols co-express endothelial and mesenchymal markers in culture. (A) Brightfield images showing changes in morphology of H7 hESC-EC in long-term culture and passaging. White scale bar, 10 µm. (B) Proliferation activity of CD31+ and CD31- populations, expressed as cell number and proliferation marker Ki67+. (C) Immunocytochemistry for endothelial marker CD31 (green) and mesenchymal marker FSP-1 (red) with Hoechst nuclei staining (blue) was performed on H7 hESC-EC, IMR90-4 hiPSC-EC and HUVEC and representative images are shown. (D) High content imaging shows cell intensity details for each cell type. FSP+/CD31- subpopulations are shown with arrows. (E) Scatter plot of real-time PCR shows a gene profile of endothelial and mesenchymal markers in the early and late cultures of hESC-derived endothelial cells generated by a 2-D differentiation protocol. Expression was determined relative to a HUVEC control and data shown are fold change mean ± SEM, n=3, note y axis in log scale. Target genes were normalised to GAPDH housekeeping genes. One-way ANOVA with Tukey’s post-hoc test: *P<0.05; **P<0.01; ***P<0.001, ****P<0.0001.

FIG. 4: VEGF is prerequisite for endothelial maintenance in hPSC-derived cells. (A) Immunocytochemistry for endothelial marker CD31 (green) and mesenchymal marker FSP-1 (red) with Hoechst nuclei staining (blue) was performed on H7 hESC-derived endothelial cells differentiated using the 3-D methods of the invention. (B) Scatter plot showing dose-dependent increase in percentage of CD31+ hESC-derived endothelial cells, total tube area and mean endothelial (CD31+) area in the populations in response to VEGF-165 (0, 5, 10 and 50 ng/ml) in the culture medium. One-way ANOVA with Tukey’s post-hoc test: ***P<0.001.

FIG. 5: Endothelial differentiation of hPSC in (A) 2D and (B) 3D cultures. Immunocytochemistry shows new CD31+ cell and vascular network formation and FSP1 supporting cells around the vascular cells from differentiation of H7 hPSC at day 12. (C) Scatter plot of real-time PCR shows a gene profile of endothelial markers during differentiation, also mean±SEM, n=4. Orange bars, 2D on Matrigel; blue bars, spinner flask without coating. Target genes were normalised to GAPDH housekeeping genes.

FIG. 6: Unique substrates metabolized exclusively by human PSC-derived endothelial cells. (A) Heat map showing different substrate utilization to Biology media M1 in CD31+ and CD31- populations from H7 hESC-EC culture, CD31+ and CD31- populations from IMR-90 hiPSC-EC, differentiated hESC at day12 and HUES hESC-EC cultures. Cells were assayed according to standard protocol and data collected after 48 hours of culture. Average height of tetrazolium reduction was measured in triplicate. (B) Dose-dependent treatment with lactose in glucose-free medium. High content imaging shows cell viability (cell number, necrotic marker Topro3 and mitochondrial membrane potential marker TMRM). Redistribution of CD31/FSP populations in hESC-EC and hiPSC-EC, shown as bar graph. Data shown are fold change mean ± SEM, n=3, One-way ANOVA with Tukey’s post-hoc test: *P<0.05; **P<0.01; ***P<0.001, ****P<0.0001.

FIG. 7: Endothelial differentiation of hPSC in 2D and 3D cultures. (A-F). Scatter plot of real-time PCR shows a gene profile of endothelial markers during differentiation, also mean±SEM, n=4. Orange bars, 2D on Matrigel; blue bars, spinner flask without coating. Target genes were normalised to GAPDH housekeeping genes. (G) Heat map. (H) bar graphs showing a comparison between 2D cultures, 3D cultures, with or without high levels of VEGF165 supplement in culture medium as well as HMVEC-c (human cardiac microvascular endothelial cells) as native cell control.

FIG. 8: Results of proteome profile angiogenesis array analysis comparing cells with regard to their angiogenic factor production. Heat map shows z score normalised levels of angiogenic factors. ECs derived from hESC (H7 hESC-EC) produced using 2D culture methods or the 3D culture method of the invention (+/- VEGF) were compared with two native EC populations: human cardiac microvascular endothelial cell (HMVEC-c) and human coronary artery endothelial cells (HCAEC), representative of the micro- and macro-vasculature beds respectively, as well as three smooth muscle cell (SMC) populations. Principal component analysis of all 54 angiogenic factors shows that 3D protocol (with VEGF165) generates cells highly similar to HMVEC.

FIG. 9: Immunohistochemistry analysis of mouse myocardial tissue following administration of endothelial cells generated in accordance with the invention. Representative image showing transplantation of hESC-derived endothelial cells generated by the invention into immunocompromised mice after myocardial infarction. Cross-section of left ventricle of the heart and epicardial presence of human cells are visualised. CD31+, green. Examples of hESC-EC on the epicardium are labelled with white arrows. 10x magnification, widefield microscopy.

FIG. 10: High content analysis of RC11 hESC-derived EC (differentiated using the 3D differentiation protocol). Representative immunocytochemistry images showing high purity population, stained for CD31 endothelial marker (green). Analysis revealed that the EC population showed consistent expression of CD31.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The publications discussed herein are provided solely for their disclosure prior to the filing date of the present application. Nothing herein is to be construed as an admission that such publications constitute prior art to the claims appended hereto.

### Endothelial Cells

As used herein the term “endothelial cell” refers to a cell which possesses specific characteristics associated with cells of the endothelial lineage. Characteristics associated with cells of the endothelial lineage have been well documented in the art and, as such, the skilled person would readily be able to test and determine whether a cell possesses these characteristics. Advantageously compared with conventional methods, the methods of the invention produce endothelial cells with a stable/maintained phenotype consistent with the native properties of mature endothelial cells. Without being bound by theory, it is believed that these properties will enhance angiogenesis and neovascularisation, and as such have therapeutic potential in the treatment of cardiovascular disease.

Typically, endothelial cells in accordance with the present invention express particular cell surface markers associated with cells of the endothelial lineage. The presence of these cell surface markers can be identified by a range of techniques available to the skilled person, including fluorescence-activated cell sorting. Cell surface markers which are characteristic of cells of the endothelial lineage include CD31 (cluster of differentiation 31, also known as platelet endothelial cell adhesion molecule (PECAM-1). Thus, preferably the endothelial cells produced by the methods of the invention are CD31⁺.

Additional characteristics associated with cells of the endothelial lineage include the expression of certain genes/proteins. Typically, endothelial cells in accordance with the present invention express at least one gene/protein which is associated with cells of the endothelial lineage. Expression of the at least one gene/protein may be increased compared to non-endothelial cells or, alternatively, may be decreased. As the skilled person will appreciate, were the expression of at least two genes/proteins is characteristic of a cell of the endothelial lineage, the expression of each of the gene/protein may independently be increased or decreased compared to non-endothelial cells. In this way, a gene/protein expression profile which is characteristic of a cell of the endothelial lineage can be produced.

Typically, endothelial cells in accordance with the present invention express at least one transcription factor, transcription regulator or DNA-binding protein. By way of non-limiting example, the endothelial cells may express one or more members of the Sox family of transcription factors, preferably an F-subgroup Sox transcription factor such as SOX7, SOX17 and/or SOX18. Other transcription factors transcription regulator or DNA-binding protein are known to the skilled person and include, for example, LYL1 (lymphoblastic leukaemia associated haematopoiesis regulator 1), YAP1 (yes-associated protein 1, also known as YAP65), HCLs1 (hematopoietic cell-specific Lyn substrate 1), HOXB3 (homeobox B3), HOXB7 (homeobox B7) and/or ZNF300 (zinc finger protein 300). An endothelial cell in accordance with the invention may express a gene/protein indicative of an active metabolic profile, for example, CYP1B1 (cytochrome P450 family 1 subfamily B member 1), PNP (purine nucleoside phosphorylase) and/or OGDH (2-oxoglutarate dehydrogenase). An endothelial cell in accordance with the invention may express a gene/protein indicative of an arterial endothelial cell phenotype, for example, APLNR (apelin receptor, also referred to as APJ), NOTCH1 (notch receptor 1) and/or Ephrin B2.

Thus, endothelial cells produced by a method of the invention may express at least one gene/protein selected from: SOX7, SOX17, SOX18, LYL1, YAP1, HCLsl, HOXB3, HOXB7, ZNF300, CYP1B1, VEGF-A, VE-Cadherin, PNP, OGDH, NOTCH1, NOTCH2, GLB1, ETV2, Ephrin B2, COL1A1, COL3A1, CD31, APLNR (also known as APJ receptor), PLAU, MMP9, ACVR1B, HGF, ERG (ETS-related gene), Tie2, Angiotensin II, ICAM2, VWF, Fli-1, ALK1, SMAD7, FSP1 and/or SMA-αβ. Typically said at least one gene/protein is expressed in addition to CD31.

Typically, the expression of at least one of SOX7, SOX17, SOX18, LYL1, YAP1, HOXB7, CYP1B1, VEGF-A, VE-Cadherin, PNP, OGDH, NOTCH1, NOTCH2, GLB1, ETV2, Ephrin B2, COL1A1, COL3A1, CD31, and/or APLNR is higher in endothelial cells generated in accordance with the invention. Typically, the expression of at least one of SOX7, SOX17, SOX18, LYL1, YAP1, HOXB7, CYP1B1, VEGF-A, VE-Cadherin, PNP, OGDH, NOTCH1, NOTCH2, GLB1, ETV2, Ephrin B2, COL1A1, COL3A1, CD31, and/or APLNR is higher in endothelial cells generated in accordance with the invention when compared with endothelial cells generated using traditional 2-D methods and/or native endothelial cells.

The endothelial cells produced by methods of the invention may be predominantly microvascular in phenotype. By way of example, the endothelial cells exhibit a predominantly cardiac microvascular phenotype. A cardiac microvascular phenotype may be determined by marker expression profile using any appropriate markers for cardiac microvascular endothelial cells known in the art. By way of non-limiting example, preferably the gene and/or protein expression of NOTCH1, NOTCH2, GLB1, ETV2, Ephrin B2 or any combination thereof is increased in endothelial cells produced by a method of the invention compared with endothelial cells generated using traditional 2-D methods and/or native endothelial cells. A cardiac microvascular phenotype may be readily determined by the skilled person, not least because human cardiac microvascular endothelial cells are well-characterised and readily available.

The endothelial cells produced by methods of the invention may be predominantly arterial in phenotype. An arterial phenotype may be determined by marker expression profile using any appropriate markers for arterial endothelial cells known in the art. By way of non-limiting example, preferably the gene and/or protein expression of: (i) APLNR; (ii) Notch 1; (iii) EphB2; (iv) APLNR and Notch1; (v) APLNR and EphB2; (vi) Notch1 and EphB2; or (vii) APLNR, Notch1 and EphB2; is increased in endothelial cells produced by a method of the invention compared with endothelial cells generated using traditional 2-D methods and/or native endothelial cells.

Typically, the expression of at least one of NRP-1, HGF and/or FSP1 is lower in endothelial cells generated in accordance with the invention. Typically, the expression of at least one of NRP-1, HGF and/or FSP1 is lower in endothelial cells generated in accordance with the invention when compared to endothelial cells generated using traditional 2-D methods and/or native endothelial cells. A decrease in expression of NRP-1, HGF and/or FSP1 or any combination thereof may be present at the same time as the expression of any of the above genes/proteins (or combination thereof) is increased.

By way of non-limiting example, endothelial cells produced by a method of the invention may express (i) low levels of HGF and/or NRP-1 (preferably both) and high levels of CD31, PLAU, MMP9 and/or ACVR1B, optionally in combination with high levels of any combination of APLNR, Notch1 and/or EphB2 as described herein.

Typically, the expression of Kinase insert domain receptor (KDR; also known as vascular endothelial growth factor receptor 2 (VEGFR-2)) is lower in endothelial cells generated in accordance with the invention compared with endothelial cells generated by traditional 2-D methods. By way of non-limiting example, endothelial cells generated in accordance with the invention are typically KDR⁻ or KDR^{lo}. The methods of the invention are able to produce a population of endothelial cells which are typically CD31⁺ and KDR⁻ or CD31⁺ and KDR^{lo-}.

The expression of additional genes/proteins may also be associated with cells of the endothelial lineage. For example, an endothelial cell in accordance with the present invention may additionally express at least one gene/protein selected from SOX7, SOX17, SOX18, LYL1, YAP1, HCLsl, HOXB3, HOXB7, ZNF300, CYP1B1, VEGF-A, VE-Cadherin, PNP, OGDH, NOTCH1, NOTCH2, GLB1, ETV2, Ephrin B2, COL1A1, COL3A1, CD31, APLNR, PLAU, MMP9, ACVR1B, HGF, ERG, Tie2, Angiotensin II, ICAM2, VWF, Fli-1, ALK1, SMAD7, FSP1 and/or SMA-αβ. An endothelial cell in accordance with the present invention may additionally express at least one gene/protein selected from EGF, NRG1, FGF4, CXCL16, IL8, FGF1, FGF7, LEP, VEGFC, TIMP4, ADAMTS1, PF4, CSF2, ANG, PROK1, PLG, CCL2, GDNF, PDGFB, TGFB1, PRL, FGF2, VASH1, IL1B, PDGFA, MMP8, TYMP, PIGF, THBS2, PSPN, SERPINB5, CCL3, ANGPT1, SERPINF1, HBEGF, PTX3, TIMP1, ARTN, IGFBP3, IGFBP1, AREG, COL18A1, EDN1, DPP4, F3, IGFBP2, THBS1, ENG, ANGPT2, SERPINE1. An endothelial cell in accordance with the present invention may additionally express at least one gene/protein selected from VE-Cadherin, Sox7, SOX17, SOX18 and/or ERG, typically in combination with CD31. Any combination of the one or more additional genes/proteins may be expressed with any one or more of those genes/proteins indicated above. By way of non-limiting example, an endothelial cell in accordance with the invention may express SOX7 and VE-cadherin. Alternatively, an endothelial cell in accordance with the invention may have high expression levels of PLAU, MMP9 and/or ACVR1B and low expression levels of HGF when compared to endothelial cells generated using traditional 2-D methods and/or native endothelial cells.

In addition to the gene/proteins recited herein, an endothelial cell in accordance with the present invention may express one or more additional gene/proteins. By analysing the expression of a number of gene/proteins, the skilled person is able to more accurately determine the phenotype of a cell.

The term “high” (or “hi”) as used herein is standard nomenclature in the art and would be readily understood by a skilled person to mean a significant increase in the expression of said marker.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or at least about a 100-fold increase, or at least about a 1,000-fold increase, or at least about a 10,000-fold increase or more, or any increase between 2-fold and 10,000-fold, e.g. between 2-fold and 10-fold, or greater as compared to a reference level. For example, an increase in expression of any given endothelial cell marker may be relative to the expression of said biomarker in the pluripotent stem cell from which the endothelial cell is derived. Alternatively, an increase in expression of any given endothelial cell marker may be relative to the expression of said biomarker in an endothelial cell produced by a conventional 2D-culture method.

In addition to positive identification markers, i.e. identification based upon the expression of a certain gene/protein and/or cell surface marker, endothelial cells may also be identified based upon a decrease in or the absence of specific characteristics. By way of non-limiting example, endothelial cells in accordance with the present invention may have decreased expression or be negative for CD14, CD45 or both, CD14 and CD45. Thus, the endothelial cells may be CD14/CD14^{lo} or CD45⁻/CD45^{lo}, preferably CD14⁻ and CD45⁻.

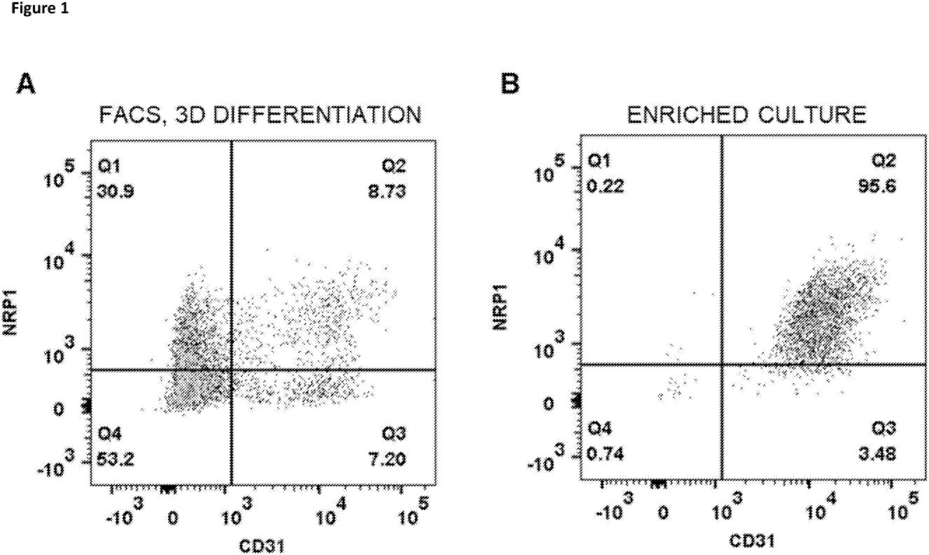

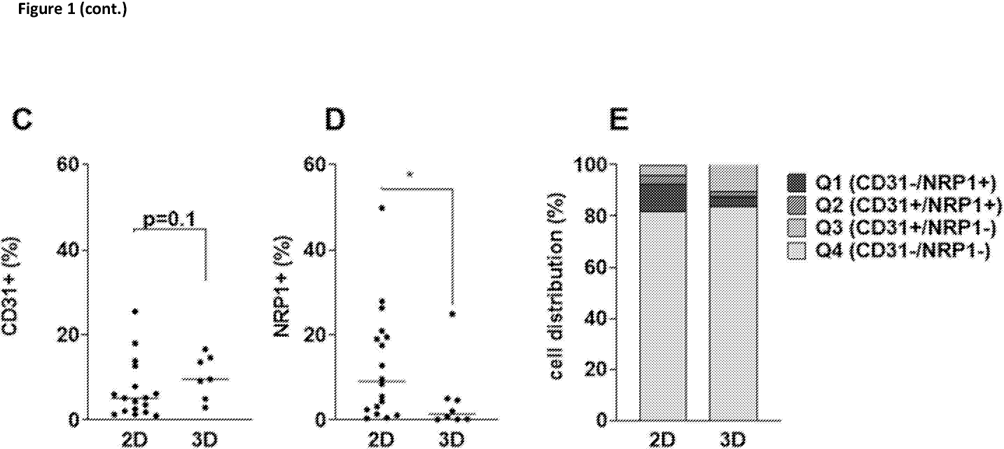

Neuropilin-1 (NRP-1, NRP1 or NP-1) is used as an endothelial cell marker in the conventional 2D-culture methods for producing endothelial cells. The present inventors have shown that NRP-1 expression levels are significantly lower in endothelial cells produced by the 3D-culture methods of the invention compared with the NRP-1 expression levels in endothelial cells produced by conventional 2D-culture methods. Accordingly, endothelial cells according to the present invention may be NRP-1⁻ or NRP-1^{lo} (as exemplified in FIG. 1). Typically, the expression of NRP-1 is lower in endothelial cells generated in accordance with the invention when compared to endothelial cells generated using traditional 2-D methods and/or native endothelial cells. The methods of the invention are able to produce a population of endothelial cells which are typically CD31⁺ and NPR-1⁻ or CD31⁺ and NRP-1^{lo-}. The methods of the invention may produce a population of endothelial cells which are (i) CD31⁺, NPR-1⁻ and KDR⁻ or KDR^{lo}; or (ii) CD31⁺, NRP-1^{lo} and KDR⁻ or KDR^{lo}.

The term “lo” as used herein is standard nomenclature in the art, and would be readily understood by a skilled person to mean a significant decrease in the expression of said marker. The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level.

Endothelial cells can also be defined in terms of their morphology. Methods of the invention generate population of endothelial cells which show increased homogeneity compared with traditional 2D culture methods. Homogeneity of a cell population may be assessed on the basis of a range of parameters known to the skilled person. Typically, endothelial homogeneity may be assessed by determining the mean cell size of the endothelial cell population and the standard deviation of the cell size of the group. A low standard deviation is indicative of a homogenous cell population. Methods of determining cell size are well known in the art. Typically, cell size may be determined using flow cytometry together with measurement of the forward scatter of the cell. Thus, in some embodiments, the standard deviation of the cell size of endothelial cells generated in accordance with the methods of the invention is lower when compared to endothelial cells generated by 2D methods.

A homogenous cell population may also be determined by assessing the percentage of endothelial cells following differentiation of a pluripotent stem cell to an endothelial cell. Typically, the cell population following differentiation of a pluripotent stem cell to an endothelial cell comprises at least 80%, at least 85%, at least 90%, at least 95%, at least 96%, at least 97%, at least 98%, at least 99% or more) endothelial cells. Thus, methods of generating a population of endothelial cells in accordance with the invention typically result in cell populations comprising at least at least 80%, at least 85%, at least 90%, at least 95%, at least 96%, at least 97%, at least 98%, at least 99% or more, up to 100% endothelial cells.

Endothelial cells can also be defined in terms of functional characteristics. By way of non-limiting example, endothelial cells are capable of forming vascular structure or tubules (capillary-like structures) in response to angiogenic signals found in conditioned media. Endothelial cells are also capable of responding to endothelial growth factors such as VEGF and platelet-derived endothelial cell growth factor (PD-ECGF). The response of endothelial cells to endothelial growth factors may be determined by assessing the expression of at least one gene/protein as described herein. Typically, the response of endothelial cells to endothelial growth factors may be determined by assessing the expression of at least one FGF4, PLAU, MMP9, ANGPT2, F3, COL18A1, IGFBP1, PDGFA, HBEGF, IGFBP2, IGFBP3, AREG and/or VEGFA, or any combination thereof. Typically, an increase in the expression of at least one of FGF4, PLAU, MMP9, ANGPT2, F3, COL18A1, IGFBP1, PDGFA, HBEGF, IGFBP2, IGFBP3, AREG and/or VEGF A, or any combination thereof, may be indicative of a response to an endothelial growth factor. In some embodiments, increased expression of a combination of FGF4, PLAU, MMP9, ANGPT2, F3, COL18A1, IGFBP1, PDGFA, HBEGF, IGFBP2, IGFBP3, AREG and VEGFA is indicative of a response to an endothelial growth factor.

Endothelial cells generated in accordance with methods of the prior art typically require co-culture, i.e. culture with a second cell type, for example fibroblasts and/or primary endothelial cells. In contrast, endothelial cells generated in accordance with the present invention are functional without co-culture. In other words, endothelial cells generated in accordance with methods of the invention exhibit the functional characteristics of endothelial cells without needing to be co-cultured alongside a second cell type. By way of non-limiting example, endothelial cells generated in accordance with the present invention may produce 3-D vascular and/or tubular structures in vitro without co-culture.

Typically, endothelial cells generated in accordance with the invention exhibit an angiogenic proteome profile. The proteome profile of a cell may be determined using a range of techniques known to the skilled person including immuno-based (for example, antibody arrays) and mass-spectrometry-based approaches. Typically, endothelial cells generated in accordance with the invention produce 3-D vascular and/or tubular structure in vitro. Vascular and/or tubular structure formation may be determined by the well-established endothelial cell tube formation assay. Thus, endothelial cells generated in accordance with the invention may exhibit increased vascular and/or tubular structure formation in vitro compared to endothelial cells generated using 2-D methods. In vivo functionality of endothelial cells may also be determined. By way of example, endothelial cells may be seeded in a 3D matrix (for example, Matrigel™) and implanted subcutaneously into mice. The ability to form a perfusable vascular network and connect with host vasculature is indicative of endothelial cell functionality. Thus, preferably, the endothelial cells are capable of forming vascular structure in vivo.

As demonstrated herein, endothelial cells produced according to the methods of the invention have a more stable endothelial cell phenotype than endothelial cells produced by the conventional 2-D methods. An “endothelial cell phenotype” is any characteristic of an endothelial cell that may be used to distinguish an endothelial cell from a non-endothelial cell. Accordingly, an endothelial cell phenotype may be defined in term of the expression of one or more endothelial cell marker (and/or absence of one or more non-endothelial cell marker), endothelial cell morphology and/or one or more functional characteristic of an endothelial cell, or any combination thereof. Any of the endothelial cell markers, morphological characteristics and/or functional characteristics described herein, either alone or in any combination, may be used to define an “endothelial cell phenotype”. Accordingly, an endothelial cell phenotype is considered stable if at least one endothelial cell characteristic (for example, at least one of those defined herein) is maintained following differentiation of a pluripotent stem cell to an endothelial cell. Accordingly, an endothelial cell phenotype may be considered stable if the expression of at least one gene/protein selected from CD31, NRP-1, SOX7, SOX17, SOX18, LYL1, YAP1, HCLsl, HOXB3, HOXB7, ZNF300, CYP1B1, VEGF-A, VE-Cadherin, PNP, OGDH, NOTCH1, NOTCH2, GLB1, ETV2, Ephrin B2, COL1A1, COL3A1, CD31, APLNR, PLAU, MMP9, ACVR1B, HGF, ERG (ETS-related gene), Tie2, Angiotensin II, ICAM2, VWF, Fli-1, ALK1, SMAD7, and/or SMA-αβ, is maintained following differentiation of a pluripotent stem cells to an endothelial cell. By way of non-limiting example, an endothelial cell phenotype may also be considered stable if: (a) high expression of any one of CD31, VE-cadherin, SOX7, SOX17 and/or SOX18 is maintained following differentiation of a pluripotent stem cell to an endothelial cell; and/or (b) low or non-detectable expression of NRP-1, HGF and/or FSP1 is maintained following differentiation of a pluripotent stem cell to an endothelial cell.

An endothelial cell phenotype may also be considered stable if the proliferative capacity of the endothelial cells is maintained following differentiation of a pluripotent stem cell to an endothelial cell. Methods of assessing the proliferative capacity are well known in the art and include, for example, the MTT cell proliferation assay and CFSE labelling.

As used herein, a “stable endothelial cell phenotype” means that an endothelial cell maintains an endothelial cell phenotype following at least one, two, three, four, five, six, seven, eight, nine, 10, 11, 12, 13, 14, 15 or more passages. Typically, a stable endothelial cell phenotype means that an endothelial cell maintains an endothelial cell phenotype for at least 3 to 12 passages. Preferably, a stable endothelial cell phenotype means that an endothelial cell maintains an endothelial cell phenotype for five to 10 passages. An stable endothelial cell phenotype may also be defined ins terms of the number of days an endothelial cell maintains an endothelial cell phenotype. For example, a stable endothelial cell phenotype means that an endothelial cell maintains an endothelial cell phenotype for at least seven days, at least eight days, at least nine days, at least 10 days, at least two weeks, at least three weeks, at least one month or more. Preferably, a stable endothelial cell phenotype means that an endothelial cell maintains an endothelial cell phenotype for at least two weeks, at least three weeks, at least one month or more. By way of non-limiting example, an endothelial cell may be considered to exhibit a stable endothelial cell phenotype if the endothelial cell is CD31⁺ for at least two weeks following differentiation, or is CD31⁺ and expresses any one of VE-cadherin, SOX7, SOX17, SOX18 and/or ERG at higher and/or any one of NRP-1, HGF and/or FSP1 at lower levels than endothelial cells generated using traditional 2-D methods and/or native endothelial cells for at least two weeks.

### Pluripotent Stem Cells

As used herein, the term “pluripotent stem cells” refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Having said that, cell pluripotency is a continuum, ranging from the completely pluripotent cell that can form every cell of the embryo proper, for example embryonic stem cells and induced pluripotent stem cells, to the incompletely or partially pluripotent cell that can form cells of all three germ layers but that may not exhibit all the characteristics of completely pluripotent cells. Pluripotent stem cells can be cells which naturally possess pluripotency, or can be cells which have been chemically or methodically made to be pluripotent (induced pluripotency). Methods of inducing pluripotency in a non-pluripotent cell (for example, an adult somatic cell) are well known in the art and typically involve the introduction of specific nuclear reprogramming substances in the form of nucleic acids or proteins into a somatic cell or by increasing expression levels of endogenous mRNAs and/or proteins of the nuclear reprogramming substances with agent(s) (K. Takahashi et al. (2007), Cell, 131(5): 861-872).

Various pluripotent stem cell types are known in the art and can be used in the methods of the invention The characteristics of pluripotent stem cells, including their marker expression (e.g. SSEA-3, SSEA-4, TRA-1-60, TRA-1-81, TRA-2-49/6E, Oct-¾, Sox2, Nanog, GDF3, REX1, FGF4, ESG1, DPPA2, DPPA4, and hTERT), chromatin methylation patterns, morphology (round shape, large nucleus with little cytoplasm, forming flat, tightly-packed colonies) and growth properties (e.g. doubling time and mitotic activity) are well-known in the art. Accordingly, it is within the routine capabilities of a skilled person to identify a pluripotent stem cell for use according to the present invention. By way of non-limiting example, pluripotency of a cell may be determined by: (i) in vitro differentiation assays (to determine the ability of the cell to give rise to all three germ layers of the body; or (ii) in vivo teratoma formation assays (whereby cells are injected into an immunocompromised animal and the ability of the cells to form a tumour comprising cells of all three germ lines is determined); (iii) chimerism of blastocytes; (iv) tetraploid complementation; and/or (v) chimera formation using a single pluripotent cell. A range of assays to determine cell pluripotency are described in Singh et al. (2016), Front. Cell Dev. Biol., 4:1-18. In addition, many different pluripotent cell types are known in the art and readily available.

The pluripotent stem cell may be an induced pluripotent stem cell (iPSC), an embryonic stem cell (ESC) a germline stem cell (GS cell), an embryonic germ cell (EG cell), an embryonic stem cell derived from a cloned embryo obtained by nuclear transfer (nuclear transfer ES cell; ntES cell), a fused stem cell, or a parthenogenetic stem cell. The pluripotent stem cell may be a cell-derived from an individual in which pluripotency has been induced, for example, using a Sendai virus vector. Pluripotent stem cell lines may also be derived from such pluripotent stem cells. Preferably, methods of the invention involve iPSCs or ESCs.

Typically, the pluripotent stem cell is a human pluripotent stem cell. In some embodiments, human pluripotent stem cell is a human pluripotent stem cell line selected from H7 hESC, RC11 hESC, IMR90-4 hiPSC and HUES7 hESC cells. Preferably, the human pluripotent stem cell is H7 hESC or IMR90-4 hiPSC cells.

### Methods of Generating Endothelial Cells

The inventors have for the first time demonstrated that endothelial cells may be generated from pluripotent stem cells in vitro in 3-D culture in the absence of non-human animal derived components. Further, the inventors have shown that such 3D-culture produces endothelial cells with a stable endothelial cell phenotype and that this method allows for the large-scale production of endothelial cells.

Accordingly, the invention provides a method of generating a population of endothelial cells from pluripotent stem cells wherein said method comprises: (a) culturing pluripotent stem cells in a 3-D suspension culture; and (b) inducing the pluripotent stem cells to undergo endothelial differentiation. The step of inducing the pluripotent stem cells to undergo differentiation into endothelial cells typically also takes place in a 3D suspension culture. Optionally said method further involves endothelial cell expansion, which preferably also takes place in a 3D suspension culture.

3-D cell culture enables cells to grow and interact with their surroundings in all three dimensions. Accordingly, 3-D culture systems more closely resemble the in vivo environment in which cells would normally grow. 3-D suspension culture is a type of 3-D cell culture in which single cells or small aggregates of cells are allowed to function and multiply in an agitated growth medium. Although suspension culture technology has been known in the art for many years, to-date there are no reports of successful suspension culture for any vascular cell type.

Typically, the 3-D suspension culture of the pluripotent stem cells is carried out in a stirred tank bioreactor. Stirred tank bioreactors are standard in the biotechnology sector and are, therefore, well known to the skilled person. The skilled person will also be aware that the bioreactor and impeller design may be adapted for a particular cell culture in order to deliver efficient mixing whilst minimising shear forces which can damage the cultured cells. Impeller speed measured in rpm (revolution per minute) may also be modulated to optimise the culture conditions. Methods of the invention typically utilise a stir-tank bioreactor operated at about 20 to 100 rpm. In some embodiments, the stir-tank bioreactor is operated at about 30 to 80 rpm, preferably at about 50 rpm.

The pluripotent stem cells may be cultured in 3-D suspension culture for at least 6 hours, at least 12 hours, at least 18 hours, at least 24 hours, at least 30 hours, at least 36 hours, at least 48 hours, at least 3 days, at least 4 days, at least 5 days, at least 6 days, at least 7 days, at least 8 days, at least 9 days, at least 10 days, at least two weeks or more prior to the step of inducing differentiation into endothelial cells. The period of time for which the pluripotent stem cells may be cultured in 3-D suspension culture prior to induction of endothelial cell differentiation may be selected to allow from expansion of the pluripotent cells to a threshold level prior to induction of endothelial cell differentiation. By way of non-limiting example, the number of pluripotent cells may be at least about 0.5 x 10⁶ cells, typically between about 0.5 ×10⁶ to about 2.0 ×10⁶ prior to induction of endothelial cell differentiation. Preferably, the number of pluripotent cells is at least about 1.5×10⁶ prior to induction of endothelial cell differentiation.

Although 3-D suspension culture of the pluripotent stem cells is central to the methods of the invention, it is envisaged that pluripotent stem cells may be culture under 2-D conditions prior to use in said methods. By way of non-limiting example, induced pluripotent stem cells are typically cryopreserved for transport or storage. Therefore, an initial step of 2-D culture of the induced pluripotent stem cells after thawing is contemplated to allow for the reestablishment and/or maintenance and/or expansion of viable pluripotent stem cells prior to use in the methods of the invention. Said 2-D culture may be carried out in coated cell culture vessels, for example cell culture vessels coated with Matrigel, particularly growth-factor reduced Matrigel. Accordingly, a method of generating endothelial cells from pluripotent stem cells according to the invention may further comprise a step of expanding the pluripotent stem cells prior to 3-D suspension culture. By “expanding the pluripotent stem cells”, the skilled person will understand this to mean increasing the number of pluripotent stem cells, typically by at least 10%, at least 20%, at least 30%, at least 50%, at least 60%, at least 70%, at least 80%, at least 90%, at least 100%, at least 150% or more, compared with the number of pluripotent stem cells before the expansion step. Preferably said expansion comprises culturing the pluripotent stem cells in a pluripotent stem cell expansion medium. Culture media suitable for the culture of pluripotent stem cells are well known in the art and include, for example, mTESR1 medium and Essential 8 medium. Typically, this pluripotent stem cell expansion step is carried out in 2-D culture, and preferably involves passaging the pluripotent stem cells when confluency of at least about 70% is achieved. Preferably any pluripotent stem cell aggregates produced by the initial 2-D expansion of the pluripotent stem cells are retained when transferring the expanded pluripotent stem cells to the 3-D suspension culture.

The 3-D suspension culture of the invention allows for the formation of pluripotent stem cell aggregates within the culture medium. The aggregate morphology permits re-establishment of the cell-cell contacts normally present in tissues; enhancing cell function and survival. The kinetics and extent of aggregation can be measured by a variety of techniques known in the art. By way of non-limiting example, direct visualisation of aggregate size may be used to determine the extent of aggregation. Aggregation kinetics can be monitored in this manner as well, by measuring aggregate size distributions over time. This procedure is facilitated by the use of computer image analysis techniques or electronic particle counters, where sometimes the disappearance of single cells (instead of the growth of aggregates) is followed. Specialized aggregometers can provide reproducible and rapid measurements of the rate of aggregation; in one such device, small angle light scattering through rotating sample cuvettes is used to produce continuous records of aggregate growth. Typically, the methods of the invention allow for the production of uniform aggregates of pluripotent stem cells. Preferably the methods of the invention enable these pluripotent stem cell aggregates to be maintained until differentiation into endothelial cells commences or is completed.

The method of generating a population of endothelial cells from pluripotent stem cells according to the invention involves the induction of said pluripotent stem cells along the endothelial lineage. Accordingly, said method comprises a step of inducing the pluripotent stem cells to undergo endothelial cell differentiation.

In accordance with the present invention, the induction of pluripotent stem cell to undergo endothelial differentiation typically comprises: (i) culturing the pluripotent stem cells in a first endothelial differentiation medium; and (ii) replacing the first endothelial differentiation medium with a second differentiation medium, wherein said first and second endothelial differentiation media are different.

An endothelial cell differentiation medium according to the invention provides one or more signal or factor that changes cell behaviour, shape, differentiation, mitotic activity, signal cascades and/or gene expression, ultimately leading to the production of endothelial cells.

In particular, an endothelial differentiation medium refers to a chemically defined cell culture medium that supports and/or enhances differentiation of pluripotent stem cells into cells of the endothelial lineage. The endothelial differentiation medium may comprise a basal culture medium supplemented with one or more agents capable of inducing endothelial differentiation of pluripotent stem cells. Various basal culture media are known in the art and include, for example, mTESR1 medium, StemLine® Haematopoietic stem cell medium, StemSpan™ Haematopoietic cell expansion media, CTS StemPro™ HSC expansion medium and StemPro™-34 SFM (serum-free medium). Preferably the medium used is not as described in WO2015/050963 or US9321995. The one or more agents, either alone, or in combination, are able to induce endothelial differentiation of pluripotent stem cells. Agents, and combination thereof, which are capable of inducing endothelial differentiation of pluripotent stem cells are well known in the art. By way of non-limiting example, the endothelial differentiation media may comprise any of the following: Activin A, BMP-4, FGF-2 and/or VEGF. The skilled person will further understand that these agents should be present in an effective amount, i.e. an amount sufficient to induce differentiation of pluripotent stem cells into endothelial cells.

Activin A is a member of the TGF-β superfamily that is known to activate cell differentiation via multiple pathways. Activin-A facilitates activation of mesodermal specification but is not critical for endothelial specification and subsequent endothelial amplification. Typically, an endothelial differentiation media comprises Activin-A at a concentration of between about 1 ng/ml to 100 ng/ml. Preferably, the endothelial differentiation media comprises Activin-A at a concentration of between about 5 ng/ml to 25 ng/ml. Even more preferably, the endothelial differentiation media comprises Activin-A at a concentration of about 10 ng/ml.

Bone morphogenetic protein-4 (BMP-4) is a ventral mesoderm inducer that is expressed in adult human bone marrow and is involved in modulating proliferative and differentiative potential of haematopoietic progenitor cells. Another member of the TGF-β superfamily, BMP-4 can additionally modulate early hematopoietic cell development in human foetal, neonatal, and adult haematopoietic progenitor cells. Typically, an endothelial differentiation media comprises BMP-4 at a concentration of between about 1 ng/ml to 100 ng/ml. Preferably, the endothelial differentiation media comprises BMP-4 at a concentration of between about 5 ng/ml to 25 ng/ml. Even more preferably, the endothelial differentiation media comprises BMP-4 at a concentration of about 10 ng/ml.

Basic fibroblast growth factor, also referred to as bFGF or FGF-2, has been implicated in diverse biological processes, including limb and nervous system development, wound healing, and tumor growth. FGF-2 has been used to support feeder-independent growth of human embryonic stem cells. Typically, an endothelial differentiation media comprises FGF-2 at a concentration of between about 1 ng/ml to 100 ng/ml. Preferably, the endothelial differentiation media comprises FGF-2 at a concentration of between about 5 ng/ml to 25 ng/ml. Even more preferably, the endothelial differentiation media comprises FGF-2 at a concentration of about 10 ng/ml.

Vascular endothelial growth factor (VEGF) is a signalling protein involved in embryonic circulatory system formation and angiogenesis. In vitro, VEGF can stimulate endothelial cell mitogenesis and cell migration. Multiple isoforms of VEGF, resulting from alternative splicing of the mRNA, have been identified including VEGF₁₆₅, VEGF₁₂₁, VEGF₁₄₅, VEGF₁₈₃, VEGF₁₈₉ and VEGF₂₀₆. Typically, an endothelial differentiation media comprises VEGF at a concentration of between about 1 ng/ml to 50 ng/ml. The concentration of VEGF may also be less than 1 ng/ml. Preferably, the endothelial differentiation media comprises VEGF at a concentration of between about 5 ng/ml to 50 ng/ml. Even more preferably, the endothelial differentiation media comprises VEGF at a concentration of about 10 ng/ml.

As the skilled person will appreciate, the concentration of each of Activin-A, BMP-4, FGF-2 and VEGF may be independently selected from the range indicated above. By way of non-limiting example, an endothelial differentiation media may comprise 12 ng/ml Activin-A, 10 ng/ml BMP-4, 8 ng/ml FGF-2 and 10 ng/ml VEGF.

The invention also encompasses the use of fragments of any of Activin-A, BMP-4, FGF-2 and/or VEGF, provided these retain the activity of the full-length molecule. Analogues, derivatives, mimetics and other small molecules which activate the same signalling pathway as any of Activin-A, BMP-4, FGF-2 and/or VEGF may also be used.

Typically, the first and second endothelial differentiation media comprises different growth factors. Preferably, the first differentiation medium comprises at least Activin-A, whilst the second endothelial differentiation medium is free of Activin A. Accordingly, the first endothelial differentiation medium may comprise Activin-A, BMP-4, FGF-2 and VEGF and the second endothelial differentiation medium may comprise BMP-4, FGF-2 and VEGF. Preferably the first endothelial differentiation medium comprises Activin-A, BMP-4, FGF-2 and VEGF, each at a concentration of about 10 ng/ml. Preferably the second endothelial differentiation medium comprises BMP-4, FGF-2 and VEGF, each at a concentration of about 10 ng/ml.

Typically, the basal medium component of the first and second endothelial differentiation media is different. The inventors have surprisingly discovered that endothelial differentiation of pluripotent stem cells in 3-D suspension culture may be enhanced by altering the basal medium component of the first and second endothelial differentiation media. Thus, the first endothelial differentiation medium may comprise a first basal medium and the second endothelial differentiation medium may comprise a second basal medium. Typically, the first basal media comprises FGF2 at higher concentrations than the second basal media. The first and second basal media may be selected form mTESR1™ or StemLine™ Haematopoietic stem cell medium. Preferably, the first endothelial differentiation medium comprises mTESR1™ medium and/or the second endothelial differentiation medium comprises StemLine Haematopoietic stem cell medium. More preferably, the first endothelial differentiation medium comprises mTESR1™ medium and the second endothelial differentiation medium comprises StemLine™ Haematopoietic stem cell medium

Typically, the induction of pluripotent stem cells to undergo endothelial differentiation comprises culturing the pluripotent stem cells in a first endothelial differentiation medium for about 6 hours, 12 hours, 18 hours, 24 hours, 30 hours, 36 hours, 42 hours or 48 hours, prior to replacing the first endothelial differentiation medium with the second differentiation medium. In a preferred embodiment, the pluripotent stem cells are cultured in a first endothelial differentiation medium for about 18 to 30 hours, more preferably about 24 hours.

The expression “replacing the first endothelial differentiation medium with a second differentiation medium” is intended to encompass a step of removing substantially all of the first endothelial differentiation medium from the cell culture and subsequently adding the second endothelial differentiation medium to the cell culture. Methods of removing cell culture media from suspension cell cultures are well known in the art. By way of non-limiting example, the cell culture (including cells and media) may be collected and centrifuged to obtain a cell pellet which can then be re-suspended in different cell culture media. Alternatively, filtration techniques, such as tangential flow filtration may be used to retain the cells in the culture vessel whilst removing the cell culture media. An adaptive feed rate (common with stirred-tank bioreactors) may be used to replace the first endothelial cell differentiation medium with the second differentiation medium. Adaptive feed rate processes are commonly employed in semi-batch cultures, for example, fed-batch cultures. When used, such adaptive feed rates are typically used from day 2 onwards.

Methods of the invention may also comprise a step of culturing the cells in the second endothelial differentiation medium. Typically, this step is performed immediately after the step of inducing the pluripotent stem cells to undergo endothelial differentiation. Pluripotent stem cells induced to undergo endothelial differentiation may be cultured in the second endothelial differentiation medium until the desired endothelial cell numbers for downstream therapeutic or experimental applications are reached. Typically, the pluripotent stem cells induced to undergo endothelial differentiation are cultured in the second endothelial differentiation medium for between 1 to 30 days. In some embodiments, the pluripotent stem cells induced to undergo endothelial differentiation are cultured in the second endothelial differentiation medium for between 10 to 25 days, preferably about 10 to 15 days, even more preferably about 12 days. The skilled person will understand that during cell culture, the cell culture media should be replaced or supplemented, typically about every 1 to 2 days during culture, in order to avoid the build-up of cellular metabolites (which can be toxic to cells) and provide fresh nutrients to the cells. Alternatively, the culture of the endothelial cells immediately after the step of inducing the pluripotent stem cells to undergo endothelial differentiation is carried out in a different endothelial cell culture medium (which may be referred to interchangeably as an endothelial cell expansion culture medium or a endothelial cell maintenance culture medium). This additional culturing step may be used to expand the number of endothelial cells produce to facilitate the production of the large numbers of endothelial cells required for therapeutic applications. By “expanding the endothelial cells”, the skilled person will understand this to mean increasing the number of endothelial cells, typically by at least 10%, at least 20%, at least 30%, at least 50%, at least 60%, at least 70%, at least 80%, at least 90%, at least 100%, at least 150% or more, compared with the number of endothelial cells before the expansion step.

In methods involving a step of culturing the cells in the second endothelial differentiation medium, the second endothelial differentiation medium typically comprises additional VEGF supplementation. By way of non-limiting example, a step of culturing the cells in the second endothelial differentiation medium may employ a second endothelial differentiation medium comprising up to 200 ng/ml VEGF. Typically, the concentration of VEGF in the second differentiation medium is between about 1 to 200 ng/ml. Preferably, the second endothelial differentiation media comprises VEGF at a concentration of between about 20 ng/ml to 100 ng/ml. Even more preferably, the endothelial differentiation media comprises VEGF at a concentration of about 60 ng/ml.

The culture vessel used for the step of culturing the pluripotent stem cells in 3-D suspension culture and/or the culture vessel used for the step of inducing the pluripotent stem cells to undergo endothelial cell differentiation and/or the additional culture step to expand the number of endothelial cells (preferably all three culture vessels), is typically feeder-cell free and/or coating-free, preferably both feeder-cell free and coating free. Feeder-cell free and/or coating-free, preferably both feeder-cell free and coating free culture vessels are preferred for application where GMP standards must be met, e.g. for endothelial cells for therapeutic applications.

Similarly, in order to meet GMP standards: (i) the expansion of pluripotent stem cells; (ii) the 3-D suspension culture of pluripotent stem cells; (iii) the induction of endothelial cell differentiation; (iv) the expansion or maintenance of endothelial cells; or (v) any combination thereof is typically carried out in xeno-free culture conditions. Typically, (i) the expansion of pluripotent stem cells; (ii) the 3-D suspension culture of pluripotent stem cells; (iii) the induction of endothelial cell differentiation; (iv) the expansion or maintenance of endothelial cells; or (v) any combination thereof is carried out in serum-free conditions, preferably non-human animal serum free conditions, even more preferably in conditions lacking both human and non-human animal serum. Preferably, (i) the expansion of pluripotent stem cells; (ii) the 3-D suspension culture of pluripotent stem cells; (iii) the induction of endothelial cell differentiation; (iv) the expansion or maintenance of endothelial cells; or (v) any combination thereof is carried out in the absence of non-human serum albumin. Typically (i) the expansion of pluripotent stem cells; (ii) the 3-D suspension culture of pluripotent stem cells; (iii) the induction of endothelial cell differentiation; (iv) the expansion or maintenance of endothelial cells; or (v) any combination thereof is carried out using media that is free from non-human animal derived components, and more preferably is carried out using human serum albumin as the only animal derived protein in the culture medium. By way of non-limiting example, (i) the expansion of pluripotent stem cells; (ii) the 3-D suspension culture of pluripotent stem cells; (iii) the induction of endothelial cell differentiation; (iv) the expansion or maintenance of endothelial cells; or (v) any combination thereof is carried out without the use of Matrigel®. In order to meet GMP standards: (i) the expansion of pluripotent stem cells; (ii) the 3-D suspension culture of pluripotent stem cells; (iii) the induction of endothelial cell differentiation; (iv) the expansion or maintenance of endothelial cells; or (v) any combination thereof is typically carried out in defined or semi-defined culture conditions, including the use of defined or semi-defined culture medium.

An adaptive feed rate may be used to reduce the concentration of lactate during (i) the 3-D suspension culture of pluripotent stem cells; (ii) the induction of endothelial cell differentiation; (iii) the expansion or maintenance of endothelial cells; or (iv) any combination thereof. High lactate is one of the key waste metabolites and usually associated with adverse culture performance and lower cell proliferation. Thus, by using an adaptive feed rate which is a well-defined production run, the methods of the invention produce endothelial cell populations with higher reproducibility and lower variability. An adaptive feed rate is typically used to reduce the concentration of lactate below about 12 mmol/L, 11 mmol/L, 10 mmol/L, 9 mmol/L, 8 mmol/L, 7 mmol/L or 6 mmol/L. Preferably, an adaptive feed rate is used to reduce the concentration of lactate below about 8 mmol/L. Alternatively, a maximum lactate concentration of about 5 g/L may be maintained.

Using methods of the invention it is possible to generate endothelial cells which exhibit a more stable endothelial cell phenotype compared to the prior art methods of generating endothelial cells, as described herein. References herein to “maintaining” or “maintenance of” endothelial cells refers to maintenance of an endothelial cell phenotype (as described herein) unless stated otherwise.

The methods of the invention can be used to produce large numbers of endothelial cells, and the yield of differentiated endothelial cells is typically greater than that obtained using a conventional 2-D culture method. By way of example, the methods of the invention can be used to produce at least about, 30 x 10⁶, 40 x 10⁶, 50 x 10⁶, 60 x 10⁶, 70 x 10⁶, 80 x 10⁶, 90 x 10⁶, 100 x 10⁶, 500 x 10⁶ or 1,000 x 10⁶ cells per batch. Typically, methods of the invention can be used to produce at least about 40 x 10⁶ to 60 x 10⁶ cells per batch, preferably about 50 x 10⁶ cell per batch. Any desired batch size may be used, with bioreactors of 100 L, or even 1000 L or more being readily available. Typically, 3-D suspension cultures are performed in batch sizes of about 5 L, about 10 L, about 20 L, about 30 L, about 40 L, about 50 L, about 100 L or more. Cell numbers achievable using methods of the invention are at least around ten times greater than those achievable using traditional 2-D culture methods. This number can be further increased by isolating and/or expanding the endothelial cells as described herein.

The methods of the invention are significantly more efficient than the methods of producing endothelial cells described in the prior art. By way of non-limiting example, the methods of the invention are able to produce endothelial cells with high levels of CD31 expression in a shorter period of time than methods of the prior art. Typically, CD31 expression of endothelial cells generated in accordance with the methods of the invention at least 10-fold, at least 25-fold, at least 50-fold, or at least 100-fold greater by day 12 of the differentiation protocol compared to undifferentiated pluripotent stem cells.

The methods of the invention may also provide other advantages, for example improved endothelial cell viability, maintained proliferative capacity of said endothelial, homogenous endothelial cell populations and/or stable endothelial cell phenotype (as described herein). Methods of the invention also provide endothelial cells which exhibit a microvascular or arterial endothelial cell phenotype. The inventors have also demonstrated that the present methods are scalable for the production of sufficient numbers of endothelial cells for cell therapy applications.

### Isolating Endothelial Cells

The methods of generating endothelial cells from pluripotent stem cells of the invention results in the production of a differentiated cell culture. As used herein, the term “differentiated cell culture” (interchangeably referred to herein as a “differentiated cell population”) is used to mean the population of cells produced by the step of inducing endothelial cell differentiation. Typically, such a “differentiated cell culture” is a population of cells in which the majority (e.g. at least 80%, at least 85%, at least 90%, at least 95%, at least 96%, at least 97%, at least 98%, at least 99% or more) of the pluripotent stem cells have differentiated. Thus, a differentiated cell culture of the invention typically comprises at least 80%, at least 85%, at least 90%, at least 95%, at least 96%, at least 97%, at least 98%, at least 99% or more, up to 100% endothelial cells. Preferably, a differentiated cell culture of the invention comprises at least between 90% to 95% or more endothelial cells. Even more preferably, a differentiated cell culture of the invention comprises at least 93% or more endothelial cells.

In order to produce a pure population of pluripotent stem cell-derived endothelial cells, the methods of the invention may further comprise a step of isolating one or more endothelial cell from the differentiated cell culture. Any appropriate means may be used to isolate the one or more endothelial cells. For example, the differentiated cell culture may be cultured in a cell culture medium which comprises one or more additive (e.g. growth factor or other supplement) which promotes the growth and/or survival of endothelial cells over any non-endothelial cell types. Examples of suitable media (sometimes referred to as endothelial cell selection media) are known in the art. Typically, techniques such as FACS, immunoprecipitation or magnetic-activated cell sorting (MACS) may be used.

The present inventors have found that there are certain carbohydrates that, out of the cells present in the differentiated cell culture of the invention, can only be metabolised by endothelial cells. Therefore, the step of isolating one or more endothelial cell may comprise or consist of culturing the differentiated cell culture with a cell culture medium comprising a carbohydrate exclusively metabolised by human endothelial cells. By including said isolation step, a substantially pure population of pluripotent stem cell-derived endothelial cells may be produced.

This technique of isolating endothelial cells can be applied to other differentiated cell populations comprising one or more endothelial cells produced by other methods, e.g. differentiated cell population comprising endothelial cells as produced by a conventional 2-D culture method. Accordingly, the invention provides a method of isolating an endothelial cell from a differentiated cell culture, comprising culturing a differentiated cell culture with a cell culture medium comprising a carbohydrate exclusively metabolised by endothelial cells. Again, a substantially pure population of pluripotent stem cell-derived endothelial cells may be produced using said method.

Whether included as a step in a method of the invention or as a stand-alone method, preferably, said carbohydrate may be selected from lactose, meso-tartaric acid, dextrin, maltotriose, D-turanose, inosine and/or alpha-keto-glutaric acid, or any combination thereof. Typically, the concentration of said carbohydrate is between about 1 mM to 5 mM, for example, about 1 mM, 2 mM, 3 mM, 4 mM or 5 mM.

Typically, methods of isolating an endothelial cell from a differentiated cell culture, comprise culturing a differentiated cell culture with a cell culture medium comprising a carbohydrate exclusively metabolised by endothelial cells for between about 1 to 5 days, for example, 1 day, 2 days, 3 days, 4 days or 5 days.

The method or step of isolating one or more endothelial cell as described herein may use a cell culture medium (also referred to as a selection or isolation medium) which comprises one or more additional additive which further promotes the isolation of one or more endothelial cell. Non-limiting examples of suitable additives include: (a) VEGF, optionally at a concentration of from about 1 to 100 ng/ml, preferably about 50 ng/ml; (b) a STAT3 inhibitor (e.g. AG490 or C188-9); and/or (c) heparin, optionally at a concentration of from about 1 to 250 mg/ml, preferably about 100 mg/ml; or any combination of VEGF, a STAT3 inhibitor and/or heparin.

The term “substantially pure” will be understood to mean a population of endothelial cells which is at least 90%, at least 95%, at least 96%, at least 97%, at least 98%, at least 99% pure, or more, up to 100% pure, i.e. at least 90%, at least 95%, at least 96%, at least 97%, at least 98%, at least 99% or more of the cells in the population are endothelial cells. Accordingly, a substantially pure population of endothelial cells will comprise less than 10%, less than 5%, less than 4%, less than 3%, less than 2%, less than 1%, or less, down to 0% non-endothelial cells.

The 3D differentiation protocol of the invention produces endothelial cells with high homogeneity (i.e. a homogenous population of endothelial cells). Thus, typically endothelial cells may be isolated from the differentiated cell culture using only a single isolation step. The isolation step may involve any isolation method described herein. By way of example, the endothelial cells may be isolated by FACS, immunoprecipitation, MACS or by culturing the differentiated cell culture with a cell culture medium comprising a carbohydrate exclusively metabolised by human endothelial cells.

### Maintaining And/or Expanding Endothelial Cells

The methods of the invention may involve one or more further steps to maintain and/or expand the resultant endothelial cells whilst retaining their endothelial cell phenotype Such a step is typically included after the isolation/purification of the one or more endothelial cells.

As discussed herein, the present inventors have found that there are certain carbohydrates that, out of the cells present in the differentiated cell culture of the invention, can only be metabolised by endothelial cells. Therefore, the step of maintain/expanding one or more endothelial cell may comprise or consist of culturing the differentiated cell culture with a cell culture medium comprising a carbohydrate exclusively metabolised by human endothelial cells.

This technique for maintaining/expanding endothelial cells whilst maintaining their endothelial cell phenotype can be applied to isolated endothelial cell populations produced by other methods, e.g. differentiated cell population comprising endothelial cells as produced by a conventional 2-D culture method. Accordingly, the invention provides a method of maintaining an endothelial cell phenotype, comprising culturing one or more endothelial cell with a cell culture medium comprising a carbohydrate exclusively metabolised by endothelial cells. Again, a substantially pure population of pluripotent stem cell-derived endothelial cells may be produced using said method (the term “substantially pure” being defined as above in relation to isolating endothelial cells).

Whether included as a step in a method of the invention or as a stand-alone method, preferably, said carbohydrate may be selected from lactose, meso-tartaric acid, dextrin, maltotriose, D-turanose inosine and/or alpha-keto-glutaric acid, or any combination thereof. Typically, the concentration of said carbohydrate is between about 1 mM to 5 mM, for example, about 1 mM, 2 mM, 3 mM, 4 mM or 5 mM.

Typically, methods of maintaining an endothelial cell phenotype, comprising culturing one or more endothelial cell with a cell culture medium comprising a carbohydrate exclusively metabolised by endothelial cells for between about 1 to 5 days, for example, 1 day, 2 days, 3 days, 4 days or 5 days.

The method or step of maintaining and/or expanding one or more endothelial cell as described herein may use a cell culture medium (also referred to as a maintenance or expansion medium) which comprises one or more additional additive which further promotes the maintenance/expansion of one or more endothelial cell. Non-limiting examples of suitable additives include: (a) VEGF, optionally at a concentration of from about 1 to 100 ng/ml, preferably about 50 ng/ml; (b) a STAT3 inhibitor (e.g. AG490 or C188-9); and/or (c) heparin, optionally at a concentration of from about 1 to 250 mg/ml, preferably about 100 mg/ml; or any combination of VEGF, a STAT3 inhibitor and/or heparin.

Methods of the invention comprising steps of maintaining and/or expanding endothelial cells can be used to further increase the number of endothelial cells produced. The methods developed by the present inventors can produce significantly increased numbers of endothelial cells as compared to existing approaches. Advantageously, methods of the invention allow for the production of sufficient numbers of endothelial cells for downstream cell therapy applications.

The endothelial cell phenotype maintained by said method or method step may be any endothelial cell phenotype described herein. By way of non-limiting example, said endothelial cell phenotype comprises expression of CD31⁺ and NRP-1^{lo}. In addition, the endothelial cell phenotype maintained by said method or method step may be stabilised (as defined herein). By way of non-limiting example, said endothelial cell phenotype may be maintained for at least one, two, three, four, five, six, seven, eight, nine, 10, 11, 12, 13, 14, 15 or more passages. Typically, said endothelial cell phenotype is maintained for at least three to 12 passages. Preferably, said endothelial cell phenotype is maintained for at least five to ten passages.

In method involving maintaining the endothelial, the cell culture medium may comprise additional VEGF supplementation. By way of non-limiting example, maintaining the endothelial cells in cell culture medium may employ a cell culture medium comprising up to 200 ng/ml VEGF. Typically, concentration of VEGF in second differentiation medium is between about 1 to 200 ng/ml. Preferably, the second endothelial differentiation media comprises VEGF at a concentration of between about 20 ng/ml to 100 ng/ml. Even more preferably, the endothelial differentiation media comprises VEGF at a concentration of about 60 ng/ml.

### Endothelial Cells and Compositions Thereof

As described herein, endothelial cells produced by the 3-D suspension culture methods of the invention differ from those produced by conventional 2-D culture methods. In particular, and as described herein, endothelial cells produced by the 3-D suspension culture methods of the present invention have a different marker expression profile compared with endothelial cells produced by conventional 2-D culture methods. For example, endothelial cells produced by the 3-D suspension culture methods of the invention are CD31⁺, NRP-1^{lo}, whereas endothelial cells produced by conventional 2-D culture methods are CD31⁺, NRP-1^{hi}.

Accordingly, the invention provides an endothelial cell, preferably a human endothelial cell, obtainable by a 3-D suspension culture method of the invention. Said endothelial cell typically has a recognisable phenotype which distinguishes it from endothelial cells produced by conventional 2-D culture methods. For example, endothelial cells produced by the 3-D suspension culture methods of the invention are CD31⁺, NRP-1^{lo}. Endothelial cells of the invention may further express one or more genes/proteins selected from SOX7, SOX17, SOX18, LYL1, YAP1, HCLsl, HOXB3, HOXB7, ZNF300, CYP1B1, VEGF-A, VE-Cadherin, PNP, OGDH, NOTCH1, NOTCH2, GLB1, ETV2, Ephrin B2, COL1A1, COL3A1, CD31, APLNR, PLAU, MMP9, ACVR1B, HGF, ERG, Tie2, Angiotensin II, ICAM2, VWF, Fli-1, ALK1, SMAD7, and/or SMA-αβ. Typically, endothelial cells of the invention express at least one of SOX7, SOX17, SOX18, LYL1, YAP1, HOXB7, CYP1B1, VEGF-A, VE-Cadherin, PNP, OGDH, NOTCH1, NOTCH2, GLB1, ETV2, Ephrin B2, COL1A1, COL3A1, and/or APLNR at higher levels than endothelial cells generated using traditional 2-D methods and/or native endothelial cells. Typically, endothelial cells of the invention express at least one of HGF and/or FSP1 at lower levels than endothelial cells generated using traditional 2-D methods and/or native endothelial cells. An endothelial cell produced by a method of the invention may have a marker expression profile comprising any combination of upregulated and/or downregulated markers as described herein (particularly in the above section on endothelial cells).

The invention further provides a composition comprising a endothelial cell of the invention, preferably a human endothelial cell, and optionally a pharmaceutically acceptable excipient. As described herein, the methods of the invention allow for the production of large numbers of endothelial cells that cannot be produced by conventional methods. Accordingly, a composition of the invention may comprise a therapeutically effective number of endothelial cells. The skilled person will readily be able to determine the appropriate number of cells which may be considered therapeutically effective.

One or more endothelial cell of the invention (as defined above) can be combined or administered in addition to a pharmaceutically acceptable carrier. Alternatively, or in addition the one or more endothelial cell of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of said compositions, therapeutic formulations, medicaments and prophylactic formulations is generally by conventional routes e.g. intravenous, subcutaneous, intraperitoneal, or mucosal routes. The administration may be by parenteral injection, for example, a subcutaneous, intradermal or intramuscular injection.

Accordingly, compositions, therapeutic formulations, medicaments and prophylactic formulations of the invention may be prepared as injectables, either as liquid solutions or suspensions. The preparation may also be encapsulated in liposomes or microcapsules.

The one or more endothelial cell are often mixed with carriers, diluents, excipients or similar which are pharmaceutically acceptable and compatible with the active ingredient. Suitable excipients are, for example, water, saline, dextrose, glycerol, or the like and combinations thereof. In addition, if desired, the one or more endothelial cell may contain minor amounts of auxiliary substances such as wetting or emulsifying agents, pH buffering agents, and/or adjuvants which enhance the effectiveness, resilience or stability of the one or more endothelial cell.

Generally, the carrier, diluent, excipient or similar is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, to facilitate long term storage.

Examples of buffering agents include, but are not limited to, sodium succinate (pH 6.5), and phosphate buffered saline (PBS; pH 6.5 and 7.5).

### Therapeutic Uses of Endothelial Cells

Advantageously, methods of the invention are able to produce substantial quantities of endothelial cells in vitro under non-human animal derived component free conditions. These cells have a range of therapeutic applications such as those described herein. In other words, the methods of the invention allow for the scalable production of large numbers of endothelial cells in compliance with GMP standards and that are suitable for therapeutic application, particularly for cell therapy and/or transplantation.

Endothelial cells form the inner lining of blood vessel and provide an anticoagulant barrier between the vessel wall and blood. As such, endothelial cells of the invention may be particularly useful for the vascularisation of injured or ischaemic tissue in a subject. More specifically, the endothelial cells of the invention may be useful in treatment of tissue injury associated with cardiovascular disease vascular disease, or ischaemic disease.

Thus, in some embodiments there is provided an endothelial cell or composition of the invention for use in the treatment or prevention of cardiovascular disease.

The invention also provides the use of an endothelial cell or composition of the invention in the manufacture of a medicament for the treatment or prevention of cardiovascular disease.

The invention also provides a method of treating or prevention of cardiovascular disease comprising administering one or more endothelial cell of the invention, or a composition of the invention to a subject in need thereof.

The term “cardiovascular disease” encompasses a host of diseases that affect the heart and vasculature. By way of non-limiting example, the endothelial cells and compositions may be used in the treatment of myocardial infarction, heart failure, cardiac stroke, peripheral artery disease (PAD), critical limb ischemia (CLI), hypertension, atherosclerosis, aortic disease, aneurysm, and angina. Vascular disease may also affect the lung and kidneys, for example, chronic renal failure, and therefore the endothelial cell and compositions of the invention may also be useful in the treatment of such disease.

Typically, treatment of a subject having cardiovascular disease involves contacting a subject, in particular, damaged, injured or ischaemic tissue, with a therapeutically effective amount of endothelial cells or composition of the invention. As used herein “therapeutically effective amount”, refers to an amount effective to at least partially treat a subject having cardiovascular disease.

The endothelial cells and/or compositions provided herein may be administered to a subject in any manner that permits the endothelial cells to graft or migrate to an intended tissue site and reconstitute or regenerate the functionally deficient area. Endothelial cells and/or compositions of the invention may be administered subcutaneously, or parenterally including intravenous, intraarterial, intramuscular, intraperitoneally, and intranasal administration as well as intrathecal and infusion techniques.

Endothelial cells and compositions of the invention may also be used to seed or populate implants or grafts which may subsequently be administered to a subject. Such implants and grafts are well known to the skilled person and include, for example, patches, stents, shunts, artificial heart valves, and scaffolds. The implant or graft may be composed of bioresorbable materials, such a bioresorbable polymers.

An endothelial cell or composition of the invention may also be used in combination with (or, in the case of a composition, include) other agents suitable for the treatment of cardiovascular disease. Such agents may, for example, promote angiogenesis and/or vasculogenesis. Such agents are well known to the skilled person and may include, for example, acidic or basis fibroblast growth factor, transforming growth factor alpha, tumour necrosis factor, platelet-derived growth factor, VEGF and angiogenin. The agent used in combination with an endothelial cell or composition of the invention may also be an agent that promotes or assists engraftment and/or survival of the endothelial cell in vivo, for example, a growth factor or cytokine.

It is within the routine practice of a clinician to determine an effective dose of endothelial cells or a composition of the invention to treat or prevent cardiovascular disease. A clinician will also be able to determine appropriate dosage interval using routine skill. The effective dose of endothelial cells is typically sufficient for pre-vascularising myocardial constructs or for the production of vascular grafts comprising an endothelial cell layer.

### Experimental Applications of Endothelial Cells and Methods of the Invention

As described herein, the methods of the invention enable the production of quantities (numbers) of endothelial cells that are not readily producible using conventional methods. As a result, the methods of the present invention may be used to produce large numbers of endothelial cells to facilitate drug development and/or screening. In additional, large numbers of endothelial cells according to the invention have potential utility in phenotypic assays.

By way of non-limiting example, to perform a drug compound screen, endothelial cells with suitable biological properties that can be sufficiently expanded in vitro for the high-throughput screening (HTS) campaign is required. Ideally, the endothelial cells are amenable to automation of cell culture scale-up and scale-out into screening ready microwell plates. In fact, a robust and scalable cell line and in vitro cell culture process are the first of three key requirements for a successful industrial cell-based HTS campaign. This invention has two major applications for the field of regenerative medicine: first, the potential to supply large numbers of autologous cells for transplantation and, second, the derivation of disease-specific endothelial cells and panels of stem cells of diverse genetic backgrounds for drug discovery and safety screening. These unique properties provided the motivation to explore and confirm the applicability of protocols that were devised to derive long-term stably proliferative endothelial cells from hESC or hiPSC

Accordingly, the present invention provides for the use of a population of endothelial cells according to the invention for the screening of new drugs and/or for phenotypic assays. Typically said population comprises at least about 1 x 10⁵, 2 x 10⁵, 3 x 10⁵, 4 x 10⁵, 5 x 10⁵, 6 x 10⁵, 7 x 10⁵, 8 x 10⁵, 9 x 10⁵ or 10 x 10⁵ cells. By way of limiting example, typical high throughput drug screening experiments employing 96-well and 384-well microplates typically requires in the region of 3 x 10⁵ cells.

### Definitions

The headings provided herein are not limitations of the various aspects or embodiments of this disclosure.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ± 4%, ± 3%, ± 2%, ± 1%, ± 0.5%, ± 0.1%, of the numerical value of the number with which it is being used.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Unless otherwise indicated, any nucleic acid sequences are written left to right in 5′ to 3′ orientation; amino acid sequences are written left to right in amino to carboxy orientation, respectively.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User’s Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| | | |
|---|---|---|
| Amino Acid | Codons | Degenerate Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/ Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more% of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

As used herein, the term “healthy individual” refers to an individual or group of individuals who are in a healthy state, e.g. individuals who have not shown any symptoms of the disease, have not been diagnosed with the disease and/or are not likely to develop the disease (e.g. a cardiovascular disease or any specific disease described herein). Preferably said healthy individual(s) is not on medication affecting a cardiovascular disease or disorder and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia

Disclosure related to the various methods of the invention are intended to be applied equally to other methods, therapeutic uses or methods, the data storage medium or device, the computer program product, and vice versa.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Preferably the subject is a mammal, e.g., a primate, e.g., a human. The terms, “individual,” “patient” and “subject” are used interchangeably herein. A subject can be male or female, adult or juvenile.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, a subject can be one who exhibits one or more risk factors for a condition or one or more complications related to said condition or a subject who does not exhibit risk factors.

A “subject in need” of treatment for a particular condition can be a subject having that condition, diagnosed as having that condition, or at risk of developing that condition.

As used herein the term “comprising” or “comprises” is used in reference to features of products, compositions and methods of the invention, that are essential, yet open to the inclusion of unspecified elements, whether essential or not.

The term “consisting of” refers to features of products, compositions and methods of the invention as recited herein, which are exclusive of any element not recited in that description of the invention.

As used herein the term “consisting essentially of” refers to those elements required for a given invention. The term permits the presence of elements that do not materially affect the basic and novel or functional characteristic(s) of that invention.

Embodiments described herein as “comprising” one or more features may also be considered as disclosure of the corresponding embodiments “consisting of” and/or “consisting essentially of” such features.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting...” also means specifically targets.

### SEQUENCE INFORMATION

SEQ ID NO: 1: a-SMA forward primer

CAAAGCCGGCCTTACAGA

SEQ ID NO: 2: a-SMA reverse primer

AGGCCAGCCAAGCACTG

SEQ ID NO: 3: ERG forward primer

GGAGTGGGCGGTGAAAGA

SEQ ID NO: 4: ERG reverse primer

AAGGATGTCGGCGTTGTAGC

SEQ ID NO: 5: GAPDH forward primer

CAAGGTCATCCATGACAACTTTG

SEQ ID NO: 6: GAPDH reverse primer

GGGCCATCCACAGTCTTCTG

SEQ ID NO: 7: SMC forward primer

CTGGCTGCAGCTTATTGATG

SEQ ID NO: 8: SMC reverse primer

CTGAGAGAGTGGATCGAGGG

SEQ ID NO: 9: TGFb2 forward primer

GGTACCTTGATGCCATCCCGCC

SEQ ID NO: 10: TGFb2 forward primer

GCACTCTGGCTTTTGGGTTCTGCA

The present invention will now be described with reference to the following non-limiting Examples.

### EXAMPLES

### Materials and Methods

### Pluripotent Stem Cell Culture

H7 human embryonic stem cells (h7 hESCs) and (IMR 90)-4 induced pluripotent stem cells (WiCell Bank, Wi, USA) and RC11 hESC, were cultured in six-well plates (Falcon, Corning, USA) with 2 ml per well of mTeSR1 complete medium (Stem Cell Technologies INC, UK) and incubated at 37° C., 5% CO2. Culture medium was changed daily, and cells were passaged using a 1:3 or 1:6 ratio when 70-80% confluency was reached (about 4-6 days). Prior to seeding the cells, plates were coated with a 1:30 diluted Matrigel solution (Becton Dickinson-BD, UK) in KnockOut DMEM medium (Gibco, UK) and incubated for 30 minutes at 37° C., 5% CO2. Passaging protocol included a washing step with phosphate buffer saline solution without calcium and magnesium (PBS w/o Ca-Mg) (Gibco, UK), cell detachment with 1 ml per well of Versene solution (0.048 mM, 0.2 gr EDTA) (Gibco, UK) and incubation for 5 minutes at 37° C., 5% CO2. After moderate tapping of the plate to dislodge colonies, cells were resuspended in fresh mTeSR1 medium, re-plated in Matrigel-coated plates and finally incubated in 37° C., 5% CO2. Blunt-end pipette tips were used to avoid breaking PSCs aggregates which is important to the colony forming ability after re-plating.

### Thawing of hiPSCs

Cryovials were stored in liquid nitrogen. For thawing, a cryovial was immersed in a water bath (37°C) and the cell suspension diluted 1 in 10 with Essential 8 (E8) medium (Thermo Fisher Scientific) supplemented with 10 µM ROCK Inhibitor Y-27632 2HCl (Selleckchem), slowly and drop-wise to prevent osmotic shock to the cells. Human iPSC suspensions were always handled with wide orifice 1000 µL tips (Starlab group) to avoid breaking the colonies into single cells. The suspension was centrifuged at 200 g for 4 minutes and the pellet resuspended in E8 + 10 µM ROCK inhibitor. ROCK inhibitor was only supplemented when cells were thawed or passaged. The contents of 1 vial were plated on 2 wells of a 6 well-plate (Becton Dickinson), which had been previously coated with growth factor-reduced Matrigel (Corning) at 37° C. for 30 minutes. The Matrigel used consisted of a 1:50 dilution of the stock with KnockOut DMEM (Thermo Fisher Scientific). Cultures were maintained at 37° C. in a 170-300 Galaxy R CO2 humidified incubator (RS Biotech), with 5% CO2 and 21% O2.

### Cryopreservation of hiPSCs

For cryopreservation, each well of a 6 well-plate of hiPSCs was lifted off as described in 2.1.3 and was resuspended in 2 mL of mFreSR cryopreservation medium (Stem Cell Technologies). The suspension was quickly added at 1 mL per cryovial (Corning), the vials were placed at -80° C. in isopropanol (VWR) containing Mr Frosty freezing container (Thermo Fisher Scientific) overnight, and transferred to liquid nitrogen the following day.

### Maintenance and Passaging of hiPSCs

The medium was replaced with fresh E8 (Thermo Fisher Scientific) every day and the hiPSCs were passaged every 4 days. Cells were lifted off by incubating with the EDTA solution Versene (Thermo Fisher Scientific) at 37° C. for 5 minutes and resuspended in E8 + 10 µM ROCK inhibitor (Selleckchem). The cell suspension was added at a 1:16 split ratio to new plates previously coated with Matrigel.

### Cryopreservation of Pluripotent Stem Cell-Derived Endothelial Cells

Differentiated cells were maintained as described below. Healthy cell cultures were trypsinised at 8-& confluency and counted as previously described. The volume of cell suspension containing 1 x 10⁶ cells was calculated and added in Freezing medium containing 80% culture medium, 10% dimethyl sulfoxide (DMSO) (Life Technologies, UK) and 10% FBS at a total volume of 1 ml inside a 1.5 ml cryogenic vial (Thermo-Fisher Scientific, UK). Cryogenic vials were then placed inside a methanol freezing container (Thermo-Fisher Scientific, UK) to achieve a 1° C./minute rate of cooling and stored in - 80° C. for one day. The next day, cryogenic vials were transferred in liquid nitrogen tank (-196° C.) for long term storage.

### Thawing of Pluripotent Stem Cell-Derived Endothelial Cells

As required, cryogenic vials were removed from liquid Nitrogen tank and placed immediately into a 37° C. water bath and shaken gently until the sides were thawed but the centre remained frozen. Then their content was added in a 15 ml falcon tube and five parts of pre-warmed EGM-2 full medium was added drop wise and mixed gently to resuspend cells. Depending on the cell type thawed, Cell suspension was added inside the appropriate culture vessel and the appropriate extra volume of EGM-2 full medium was added. After 24 hours, cell cultures were checked under the microscope and culture media was replaced to replenish nutrients and remove any DMSO residues.

### mTESR1 Medium Composition

This complete, serum-free and feeder-free medium was prepared from the following components: DMEM F12 (no. SH30023) from Thermo Scientific; trace elements B (no. 99-175-CI) and C (no. 99-176-CI) from Cellgro; LiCI (no. L121) and thiamin (no. BP892) from Fisher Scientific; L-glutamine (no. 25030) and chemically defined lipid concentrate (no. 11905) from Life Technologies; BSA as above; pipecolic acid (no. 217616) from MP Biomedicals; and GABA (no. A5835), insulin (no. I-6634), L-ascorbic acid (no. A8960), nonessential amino acid (no. M7145), reduced glutathione (no. G4251), and sodium selenite (no. 214485) from Sigma-Aldrich. In addition, recombinant human basic fibroblast growth factor (rh bFGF) and recombinant human transforming growth factor β (rh TGFβ) were also added.

### Confocal and High Content Automated Microscopy of Endothelial Cells

For the identification and visualisation of stained cells, cultured plates were imaged using confocal microscopy (Zeiss LSM-780 inverted) and representative pictures were acquired in 20 x magnification. Furthermore, for the detection and quantification of a fluorescent signal produced by stained cells in a 96-well plate format, a strategy developed by our group using high content automated microscopy was performed (Foldes G et al, Methods Mol Biol, 2013). For this purpose, plates were scanned on ArrayScan™ VTi automated microscopy and image analysis system (Cellomics Inc., Pittsburgh, PA, USA) using modified Target Activation, Morphology Explorer (cell area and structural assessment) and Compartmental Analysis (STAT3 nuclear translocation) BioApplication protocols. Cells were identified with Hoechst which identified the nuclear area in fluorescence channel 1, CD31 in channel 2 (Alexa Fluor 488) and FSP-1 in channel 3 (Alexa Fluor 546) respectively. Fluorescence intensities of expressing cells were selected using a 10x objective and % high average intensities for each well of the plates scanned were subsequently calculated

### Example 1: Differentiation of hPSCs Into Endothelial Cells in 3-D Suspension Culture

hPSCs were detached from the maintenance culture by performing a washing step with PBS w/o Ca-Mg prior to addition of Versene solution. The cell suspension was centrifuged at 1200 rpm for 5 min with the supernatant being aspirated and the cell pellet being resuspended in 1 ml mTeSR1. Afterwards, the number of cells obtained was calculated according to the trypan blue exclusion method, using a 0.4% trypan blue solution (Gibco, UK) and a Neubauer haemocytometer (Hauser Scientific, Bright-Line, USA).

After cell counting, the volume of cell suspension containing enough cells for spinner flask bioreactors was calculated and diluted in the appropriate volume of mTeSR1 medium containing 10 ng/ml of Rho-associated protein kinase (ROCK) inhibitor (Gibco, UK). After two more days (Day 0), the media was replaced with mTeSR1 medium per well containing the growth factors Activin A (R&D systems, UK, #338-AC), bFGF (R&D systems, UK, #4114-TC), VEGF165 (Peprotech, UK, #100-20) and BMP4 (R&D systems, UK, #314-PB/CF) all at a concentration of 10 ng/ml, in order to initiate the differentiation. After 24 hours (Day 1), the medium was replaced with Stemline II Hematopoietic stem cell medium (Sigma-Aldrich, UK) containing bFGF, VEGF165 and BMP4, at a concentration of 10 ng/ml (Differentiation medium). On day 3 and 6 to 12, fresh differentiation medium was added to the culture. Cell cultures were monitored during the differentiation process and detect signs of contamination or cell death. RNA samples were collected on day 0, day 5 and day 12 to analyse gene expression profile of differentiating cells. Rotor speed, feeding regime and starting cell density was optimised for each line.

After culture in differentiation medium supplemented with growth factors, differentiated hPSCs were sorted based on CD31 and Neuropilin-1 (NRP-1) surface expression (FIG. 1) and were expanded in endothelial maintenance culture medium.

### Example 2: Comparison of CD31 and NRP1 Surface Expression of hPSC-Derived Endothelial Cells Generated Using 2-D and 3-D Protocols

Fluorescence activated cell sorting (FACS) was used to analyse endothelial cell populations produced according to example 1 (i.e. endothelial cells differentiated in 3-D suspension culture) and according to a traditional 2-D differentiation protocol (Prasain et al. Nat Biotechnol. 2014 Nov;32(11):1151-1157).

Human Embryoid Body Dissociation Kit (Miltenyi, Germany) was used to prepare endothelial cells from 3-D cultures for FACS. Cells were isolated for analysis at day 12 following differentiation.

Cells produced by 2-D culture were detached from culture plates by trypsinisation including a washing step with PBS w/o Ca-Mg followed by addition of 100µl/well of pre-warmed 0.05 M EDTA/trypsin solution (Gibco, UK) and incubation at 37° C. for 3 minutes. After incubation, moderate tapping of the plate caused the colonies to dislodge and 5 parts of 10% FBS/Stemline II medium was added per well to deactivate trypsin (5:1 ratio). The resulting cell suspension was dispersed by gentle pipetting and filtered using a 70 µm strainer (Falcon, Corning, USA) to eliminate clumps and debris. After this, cells were counted (trypan blue exclusion) to make a rough estimation about their number and viability.

Suspended cells (i.e. cells produced by 3-D culture) were centrifuged at 1200 rpm for 5 minutes and cell pellet formed was resuspended in FACS/blocking buffer 1% foetal bovine serum (FBS) (Gibco, UK) in PBS w/o Ca-Mg.

Cell samples were prepared including an unstained control, single stained controls and the full stained sample. Fluorescent antibodies (anti-human CD31 Alexa Fluor 488-conjugated (BD, UK, #557703) and NRP-1 APC-conjugated (Miltenyi Biotec, UK, #130-090- 900)) were added at a dilution rate of 1:20 and 1:11 respectively up to 1 × 10⁷ cells/ FACS buffer concentration. After addition of the antibodies, samples were mixed using a vortex in low speed for 30 seconds and then incubated at 4° C. for 25 minutes in the dark. Following the incubation, cells were resuspended in another volume of PBS w/o Ca-Mg and centrifuged again at 1200 rpm for 5 minutes to wash the excess of the antibodies that did not conjugate. Finally, each sample was resuspended in 0.5 ml of 1% FACS buffer and filtered again through a 20 µm strainer (BD, UK) to form a single cell suspension and avoid clogging the FACS analyser. Cell samples were kept in the dark at 4° C. until the scheduled time for sorting which was performed on a FACS Aria analyser (BD, UK).

For sorting the CD31⁺/NRP-1^{lo} cells, single stained controls were used to exclude non-specific antibody binding on cell surface and set up the analysis gates. Unstained sample was used as a negative control and for the elimination of samples auto-fluorescence (FIG. 1).

As shown in FIG. 1D, endothelial cell produced using the 3-D differentiation protocol of the invention had significantly reduced surface expression of NRP-1 compared to endothelial cells produced using traditional 2-D methods.

### Example 3: Analysis of Endothelial Cell Size by FACS

The cell size of hESC-derived endothelial cells generated by the traditional 2-D differentiation protocol indicated above and the 3-D differentiation protocol according to the invention was determined using FACS. FACS was used to provide a measurement of forward scatter (FSC), which allows for the discrimination of cells by size. FSC intensity is proportional to the diameter of the cell and is primarily due to light diffraction around the cell. FSC is detected by a photodiode, which converts the light into an electrical signal. Standard distribution of FSC (rSD) was also calculated and provides an indication of the homogeneity of cell size within a cellular population.

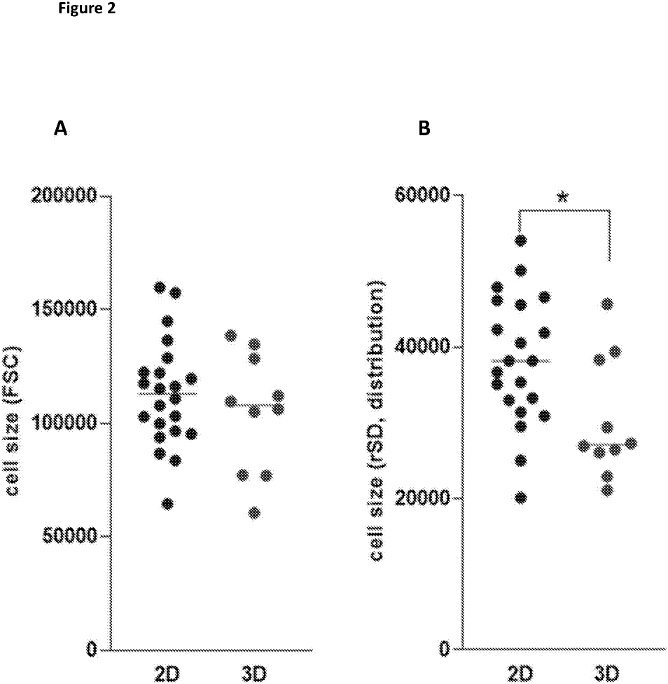

As shown in the FIG. 2 (specifically, FIG. 2B), hESC-derived endothelial cells generated using the 3-D methods of the invention are more homogenous than cells generated using 2-D methods.

### Example 4: Analysis of 2-D Differentiated Endothelial Cells

H7 hESC and IMR hiPSC were differentiated according to the traditional 2-D protocol as indicated above. H7 hESC-derived endothelial cells, IMR hiPSC-derived endothelial cells and HUVEC cells were then expanded and maintained as described in example 5 below.

### Phenotypic Analysis

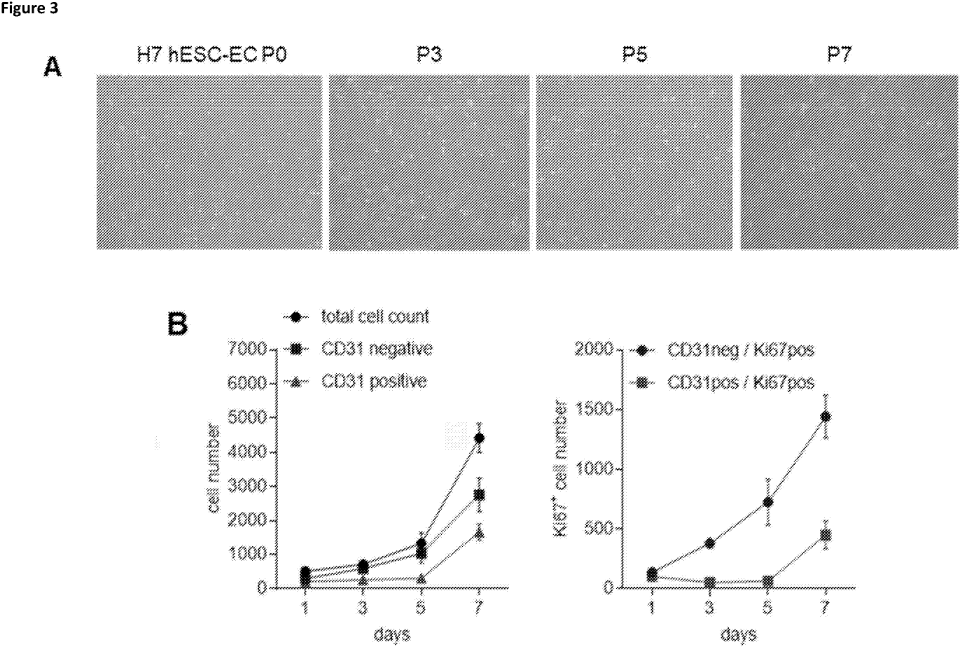

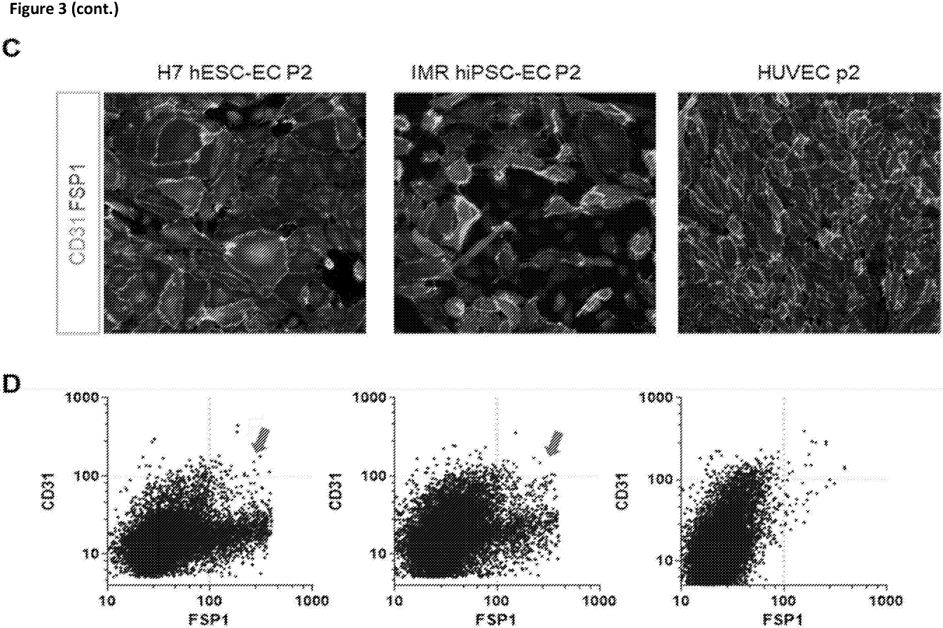

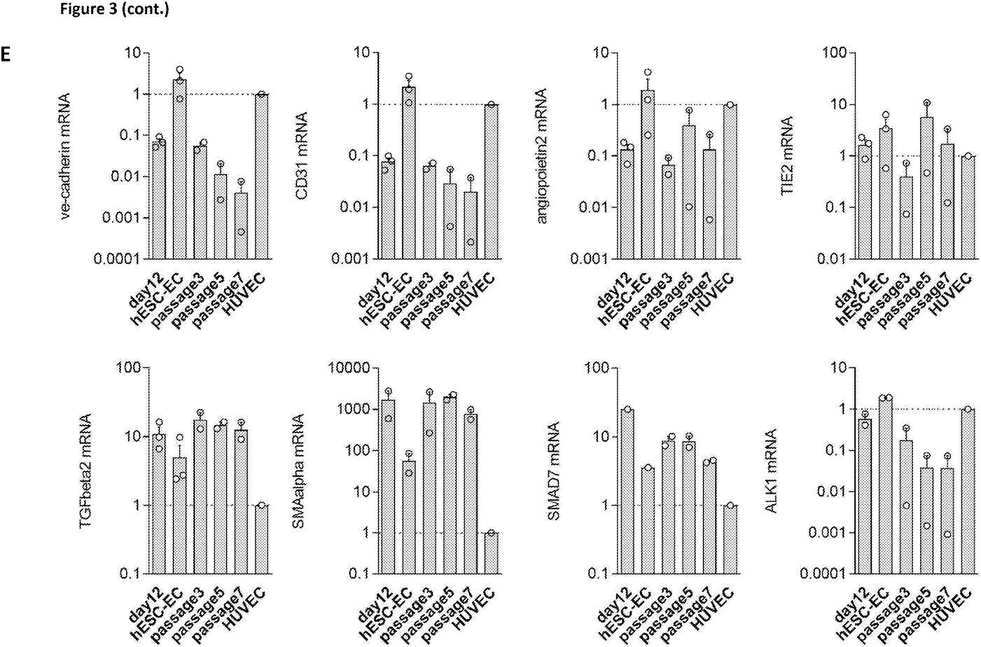

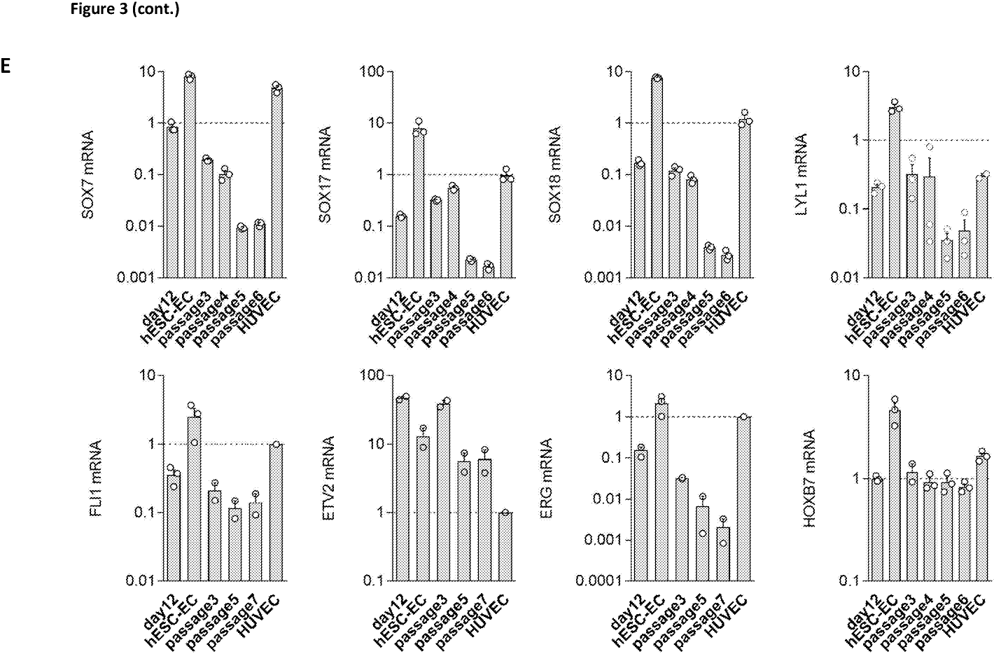

Brightfield microscopy of H7 hESC-derived endothelial cells was used to determine cellular morphology following 2-D differentiation and after 3, 5 and 7 passages (FIG. 3A). Changes in cell morphology can be seen with increasing passage number.

FACS analysis was used to determine CD31 and Ki67 expression of these cells (FIG. 3B). Proliferation activity of CD31+ and CD31- populations of these cells was determined and expressed as number of cells expressing the proliferation marker Ki67+.

Immunocytochemistry for endothelial marker CD31 (green) and mesenchymal marker FSP-1 (red) with Hoechst nuclei staining (blue) was performed H7 hESC-derived endothelial cells, IMR hiPSC-derived endothelial cells and HUVEC as described below. Representative images are shown in FIG. 3C. High content imaging was performed as described above and cell intensity details for each cell type is show in FIG. 3D.

### Gene Expression

The gene expression of native endothelial cells (HUVEC) and hESC-derived endothelial cells differentiated using the 2-D method at day 12 following differentiation, (day 12), after enrichment based on CD31 surface expression (hESC-EC) and at passage 3, 4, 5 and 6 was investigated. FIG. 3E shows the expression of a variety of endothelial and mesenchymal markers relative to HUVEC (row 1 are standard endothelial cell markers, row 2 are standard mesenchymal cell markers, rows 3 and 4 are other relevant markers).

For RNA extraction, after washing two times with a volume of PBS w/o Ca-Mg, 1 ml per 10 cm² plating surface of TriReagent solution (Sigma-Aldrich, UK) was added into cell culture and cells were incubated in room temperature for 5 minutes before being scraped. Homogenised cells were collected and transferred to a 1.5 ml Eppendorf tube (Greiner bio One, UK) and after repetitive pipetting and vortexing for 1-minute lysed samples were stored at -80° C. RNA isolation and purification were performed using the RNeasy Mini Kit (Qiagen, UK) according to manufacturer’s instructions. Briefly 70% ethanol solution was added to cell lysates which were then loaded onto the RNeasy mini spin columns provided. Lysate contaminants were removed by serial washing with the washing buffers provided and by centrifugation steps as instructed. Extracted RNA samples were eluted in 30 µl of nuclease-free water and the concentration and purity of the RNA samples were measured using a Nanodrop 8000 spectrophotometer (Thermo-Fisher scientific, UK) at 230-280 nm spectrum. Samples were stored at - 80° C. prior to further analysis.

After RNA extraction and measurement of its concentration, single stranded cDNA synthesis by reverse transcription followed using the High Capacity cDNA Reverse Transcription (RT) Kit (Invitrogen, UK, #4368814). Firstly, each RNA sample was diluted in Nuclease-free water (Gibco, UK) at a total volume of 10 µl inside 0.2 µl reaction tubes (VWR International, UK) with the dilution ratio depending on the initial RNA concentration measured. Then 10 µl of 2 X RT master mixes were prepared and added into each diluted RNA sample at a total volume of 20 µl, followed by thorough mixing using the pipette. Samples were centrifuged at a low speed for 30 seconds and then loaded to a Gene Amp(R) PCR system 2700 (Applied Biosystems, UK) where reverse transcription reaction was performed. Produced cDNA samples were stored in -20° C.

Expression of the genes of interest was quantified through RT-qPCR with every reaction performed in technical replicates (triplicates) to exclude technical errors. Expression signal was detected by fluorescent dyes, namely SYBR green and Taqman chemistries. The relative gene expression of target genes was based on the expression of the housekeeping gene Glyceraldehyde 3-phosphate dehydrogenase (GAPDH) and was determined using the 2-dct method (Kenneth J. Livak and Thomas D. Schmittgen, Methods, 2001).

CD31, VE-Cadherin, ERG, Tie2 and Ang2 expression levels were determined using Taqman PCR (CD31: Hs00169777_m1, Ang2: Hs01048043_m1, Tie2 (TEK): Hs00945142_m1, ERG: Hs01554629_m1; VE-Cadherin (CDH5): Hs00901463_m1; GAPDH: 4333764F endogenous control, Applied Biosystems, see Table 2). The level of SOX7, SOX17, SOX18, LYL1 and HOXB7 was also determined. The TaqMan Universal Mastermix II (Applied Biosystems) was used for all reactions diluted 1:2 and all assays were diluted 1:20. Samples were run on the Mastercycler RealPlex 2 (Eppendorf) Real-Time PCR system.

ETV-2, Fli-1, ALK1, SMAD7, SMA-a and TGFβ2 expression levels were determined using an SYBR Green mix (PerfeCTa SYBR green FastMix, Quanta Biosciences). The SYBR green FastMix was used for all reactions diluted 1:2 and forward and reverse primers at a concentration of 10pmol/µl were diluted 1:25. The sequences of all the primers used for SYBR green reactions are shown in the 2. Samples were run on the C1000 Thermal Cycler CFX96 Real-Time PCR system (BioRad). qPCR reactions were carried out in triplicate for all genes of interest. Relative expression was determined using the ΔΔCt method with GAPDH as the housekeeping control. Firstly, cDNA samples were diluted in nuclease free water at 1:4.5 ratio and for each reaction 4.5 µl of diluted cDNA, 0.5 µl of FAM-conjugated TaqMan probe and 5 µl of

TaqMan Gene expression MasterMix (Applied Biosystems, UK, #4369016) were mixed at a total 10 µl reaction volume. RT-qPCR Reactions were prepared in a 384-well microfluidic card (Thermo-Scientific, UK) and carried out using a 7500HT Fast Real-Time PCR System (Applied Biosystems, UK) under the appropriate amplification programme. SYBR green RT-qPCR: was used to determine the expression of a-SMA, ERG, SMC and TGFb2 genes (Table 2). Firstly, cDNA samples were diluted at 1:10 ratio while forward and reverse primers (10pmol/µl concentration) at 1:25 ratio in nuclease free water respectively. 25 µl of each reaction mixture was prepared by mixing 12.5 µl of PerfeCTa SYBRTM green FastMix (Quanta Biosciences, UK, #84067), 5 µl of diluted cDNA and 1 µl of forward and reverse primer diluted solution. RT-qPCR Reactions were prepared in a 96-well reaction plate (ThermoScientific, UK) and were carried out with an Eppendorf Realplex Mastercycler (eppendorf, UK) using the appropriate amplification programme.

TABLE 1

TaqMan probe information

| | |
|---|---|
| TaqMan | Access code |
| CD 31 (PECAM1) | Hs00169777_m1 (Thermo Fisher Scientific) |
| GAPDH | 4333764F (Thermo Fisher Scientific) |
| Oct-4 (POU5F1) | Hs00999634_gH (Thermo Fisher Scientific) |
| SOX7 | Hs00846731_s1 (Thermo Fisher Scientific) |
| SOX17 | Hs00751752_s1 (Thermo Fisher Scientific) |
| SOX18 | Hs00746079_s1 (Thermo Fisher Scientific) |
| VE Cadherin (CDH-5) | Hs00901463_m1 (Thermo Fisher Scientific) |

TABLE 2

SYBR RT-qPCR primer sequence information

| | | |
|---|---|---|
| SYBR Green | Forward primer seq. 5′- 3′ | Reverse primer seq. 5′- 3′ |
| a-SMA | CAAAGCCGGCCTTACAGA | AGGCCAGCCAAGCACTG |
| ERG | GGAGTGGGCGGTGAAAGA | AAGGATGTCGGCGTTGTAGC |
| GAPDH | CAAGGTCATCCATGACAACTTTG | GGGCCATCCACAGTCTTCTG |
| SMC | CTGGCTGCAGCTTATTGATG | CTGAGAGAGTGGATCGAGGG |
| TGFb2 | GGTACCTTGATGCCATCCCGCC | GCACTCTGGCTTTTGGGTTCTGCA |

### Example 5: Maintenance of 3-D Differentiated Endothelial Cells in VEGF Containing Media

H7 hESC-derived endothelial cells generated in accordance with example 1 were sorted using FACS as described in example 2. CD31⁺ / NRP-1^{lo} cells were collected in sterile polystyrene round bottom tubes (BD, UK) containing 1ml of 40% Stemline II medium, 40% of full supplemented endothelial growth factor medium-2 (EGM-2) (EBM-2 plus all SingleQuot supplements, Lonza, UK), 20% FBS and 1% Penicillin/Streptomycin solution (P/S) (Gibco, UK). Supplier’s information states that EGM-2 supplements include: human epidermal growth factor (hEGF), gentamicin-amphotericin-B 100, R3-insulin growth factor (IGF)-1, ascorbic acid, vascular endothelial cell growth factor (VEGF), human fibroblast growth factor (hFGF)-B and heparin, hydrocortisone. A small proportion of the sorted cells were reanalysed to check sorting efficiency and purity. 5000 cells were seeded per well onto 24 well plates coated with collagen IV (Sigma-Aldrich, UK, #C7521) and 1 ml of 50% EGM-2 medium, 50% Stemline II medium and 1% P/S. This medium was replaced with 1 ml of 75% EGM-2, 25% Stemline II and 1% P/S media after two days (day 14) and with 100% EGM-2 and 1% P/S after three more days (day 16). At this stage, sorted cells were considered as passage 0 cells. After reaching 80% confluency (around day 19-20), hPSC-derived endothelial cells were split (passage 1) by trypsinisation and counted. 10,000-12,000 cells/cm² were seeded onto a collagen IV coated T25 flask (25 cm² surface area) (Greiner bio One, UK) with 5 ml of EGM-2 medium. EGM-2 full medium was replaced every two days until cells reached 80% confluency. When confluent, cells were used for experiments.

For experiments determining the effect of VEGF on the maintenance of H7 hESC-derived endothelial cells, EGM-2 medium was prepared as indicated above, albeit, without VEGF. EGM-2 medium containing 0 ng/ml, 5 ng/ml, 10 ng/ml and 50 ng/ml VEGF₁₆₅ was also prepared. Cells were treated with the varying concentration of VEGF for 48 hours.

### Analysis of CD31 and FSP1 Expression

Analysis of CD31 and FSP1 expression of cells treated with VEGF was determined by Immunocytochemistry.

For immunocytochemistry experiments, H7 hESC-derived endothelial cells maintained as described above were seeded in 96-well plates at 5000 cells per well and treated with EGM-2 and VEGF as described.

Cells were then washed twice with PBS w/o Ca-Mg and 100 µl of 4% paraformaldehyde (Sigma Aldrich, UK) diluted in PBS w/o Ca-Mg were added per well for fixation. After incubation for 12 minutes at room temperature cells were washed again twice with a volume of PBS w/o Ca-Mg and fixed cells were permeabilised with a 0.5% (v/v) Triton X-100 (Sigma Aldrich, UK) in PBS w/o Ca-Mg solution per well for 5 minutes at room temperature. After permeabilization, cells were incubated in 4% FBS/PBS w/o Ca-Mg blocking solution for 60 minutes at room temperature. After blocking, staining with anti-human primary antibodies of interest took place. Specifically, cells were co-incubated in 3% (w/v) BSA/PBS w/o Ca-Mg containing mouse anti-human CD31 Alexa Fluor 488-conjugated (#557703, BD) and rabbit anti-human FSP1/S100A4 antibody (#07-2274, BD) in 1:50 and 1:100 dilutions respectively. Plates were incubated for 60 minutes in room temperature and in the dark. In one of the wells, only antibody diluent without primary antibody was added for checking non-specific binding (unstained negative control). After primary antibody incubation, plates were washed three times in PBS w/o Ca-Mg for 5 minutes and incubation in 3% BSA/PBS w/o Ca-Mg secondary fluorescent antibody solution for 45 minutes and in the dark followed (secondary antibodies: Alexa Fluor 546 donkey anti-Rabbit IgG (#A10040, Life Technologies, UK) and Alexa Fluor 488 donkey anti-mouse IgG (#A21202, Life Technologies, UK), both diluted1:1000). After secondary antibody incubation, wells were washed 3 times in PBS w/o Ca-Mg for 5 minutes and incubation with a 3% BSA/PBS w/o Ca-Mg Hoechst nuclear dye solution (1:200 dilution) (#33342, Life Technologies, UK) was performed for 10 minutes and in the dark. After incubation, cells were washed three times in PBS w/o Ca-Mg for 5 minutes and stored in the dark.

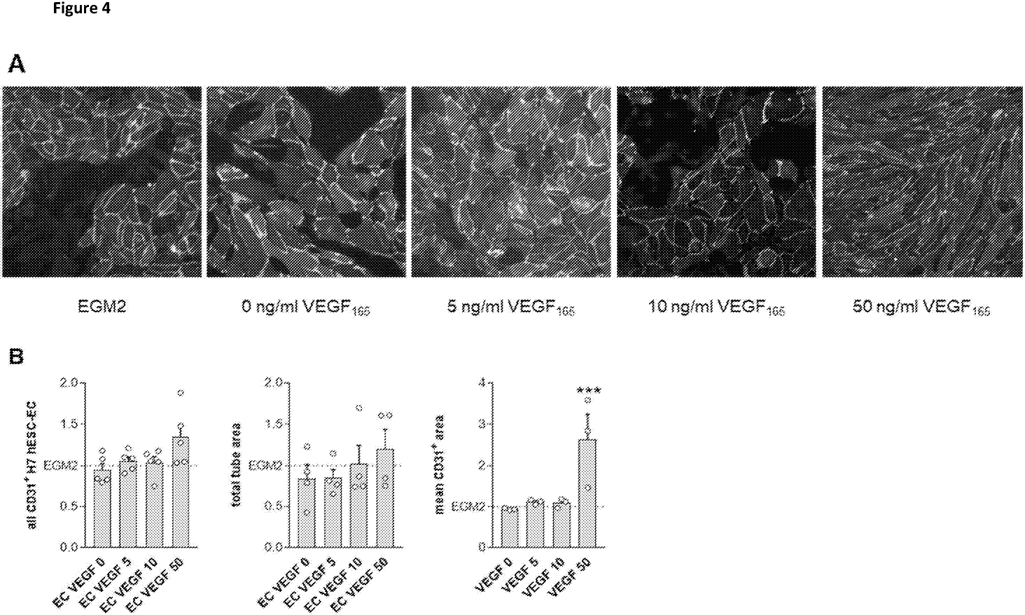

Confocal and high content microscopy was performed as described in the methods above. CD31+ cells were counted and the mean CD31+ area was determined (FIG. 4).

### Matrigel Tube-Formation Assay

Growth factor-containing undiluted Matrigel was thawed overnight at 4° C. on ice. 48 well plates were then coated with 130 µl Matrigel per well. H7 hESC-derived endothelial cells as described were seeded at 30,000 cells per well onto Matrigel that had solidified for one hour at 37° C. Images were taken at 6- and 24-hours following seeding. Each condition was performed in duplicate. Quantification of total tube area was performed using ImageJ (FIG. 4).

### Example 6: Comparison of Endothelial Cells Differentiated With 2-D and 3-D Protocols

Cells were differentiated according to the traditional 2-D protocol and the 3-D protocol of the invention as described.

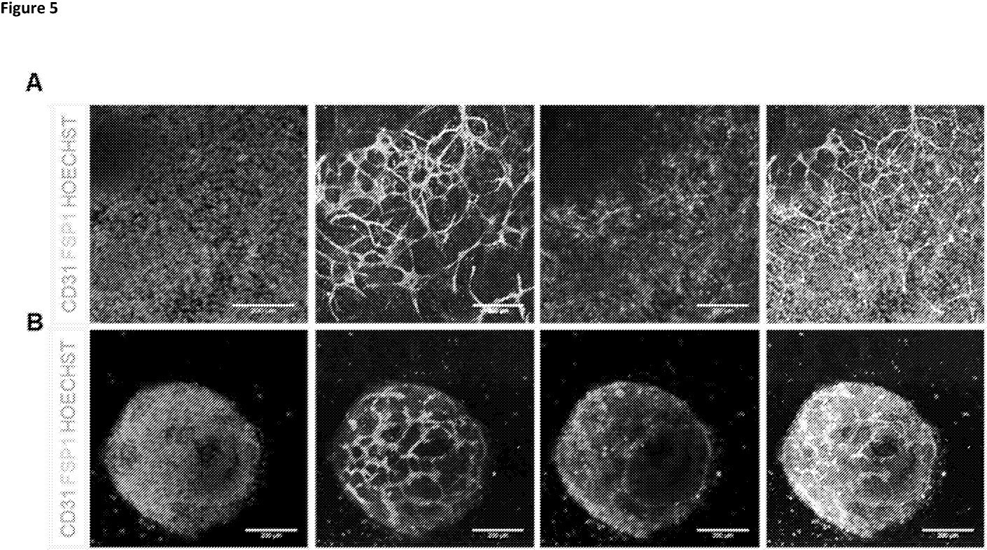

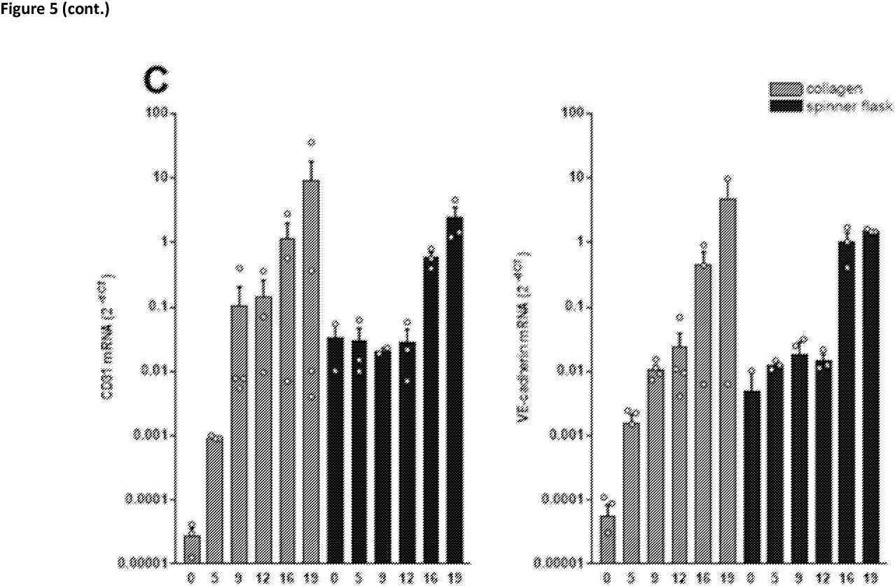

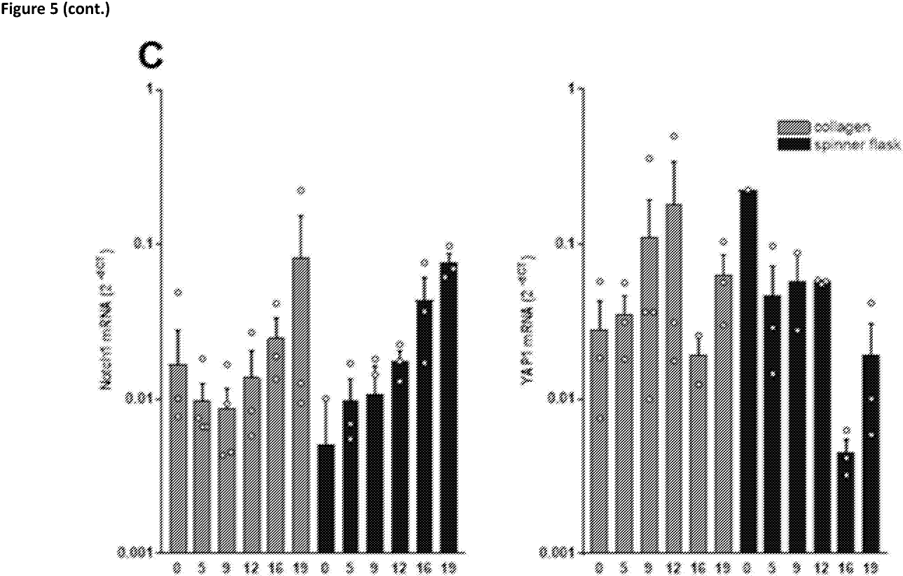

Immunocytochemistry to assess CD31 and FSP1 expression was carried out at day 12 of culture. Confocal microscopy imaging revealed that endothelial cells generated according to the 3-D method of example 1, exhibit vascular network formation in the absence of exogenous extracellular matrix, for example Matrigel. In contrast, an exogenous extracellular matrix was required for vascular network formation by the endothelial cells producing using the traditional 2-D protocol (FIG. 5A and B).

RNA was extracted from cell cultures at day 0 (differentiation), and at days 0, 5, 9, 12 and 19 of culture and gene expression was analysed as described in example 4. The expression of CD31, VE-cadherin, Notch1 and YAP1 was determined for the 2-D method (collagen) and the 3-D method (spinner flask) at each time point. The results are show in FIG. 5C.

### Example 7: Analysis of Substrate Utilisation and Maintenance of Endothelial Cells

The utilisation of a range of metabolic substrates was determined for a variety of endothelial cells using a metabolic selection plate. H7 hESC-derived endothelial cells (H7 CD31+ day19), IMR hiPSC-derived endothelial cells (IMR CD31+ day19) and HUES7 hESC-derived endothelial cells (HUES7 CD31+ day19) were all generated according to example 1 (i.e. by the 3-D differentiation protocol of the invention. CD31- cells (which have a mesenchymal phenotype) were employed as a comparison (H7 CD31- p6, H7 CD31- day12 and IMR CD31- p5).

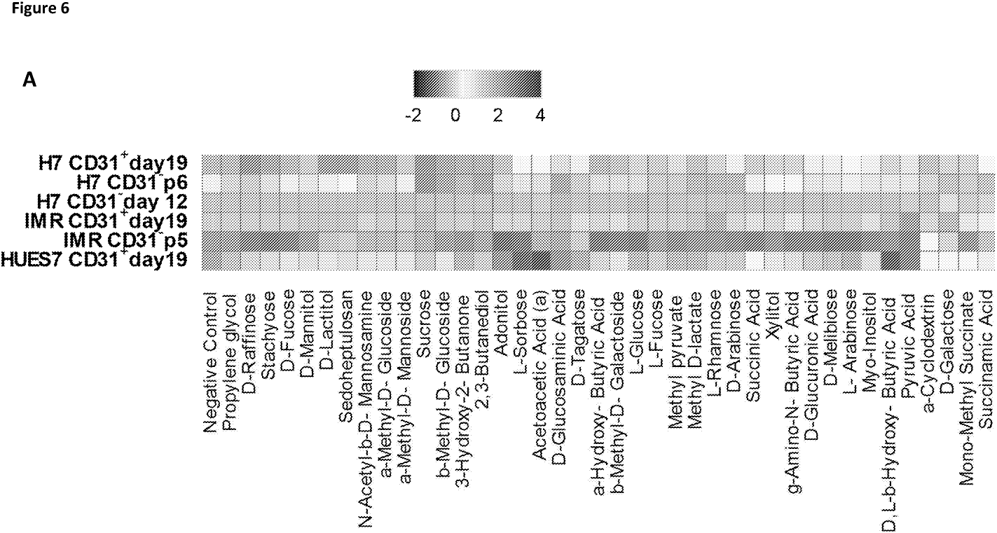

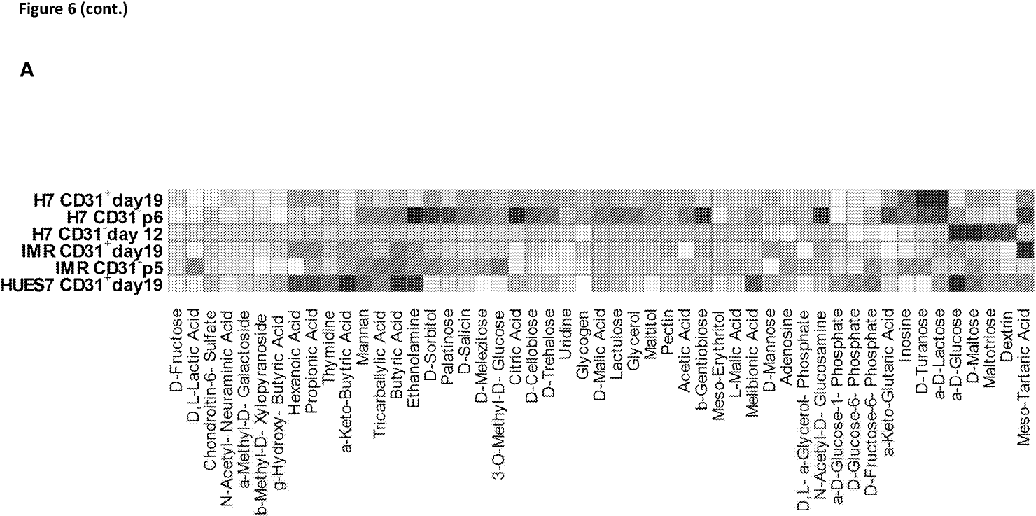

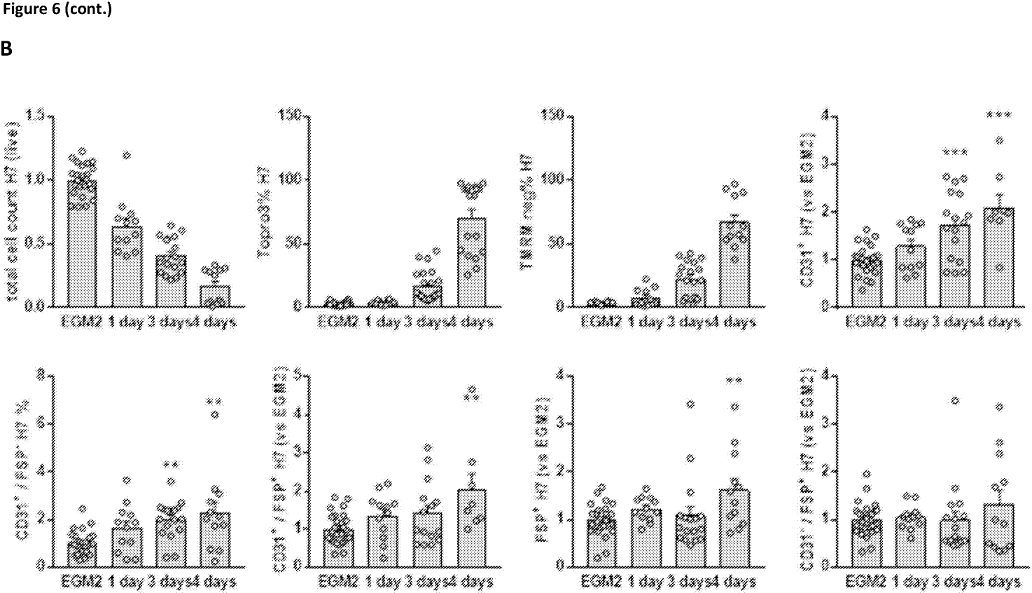

For metabolic substrate utilisation experiments, cells were seeded into 96 well plates (TPP, Trasadigen, Switzerland) at seeding densities of 10.000/well in a complete medium MC-0 (50 µL/well). The MC-0 medium was prepared using either IF-M1 medium with Pen/Strep, 0.3 mM L-glutamine and 5% FBS (Lonza, Basel, Switzerland). Metabolic capability (by measuring tetrazolium reduction) was assessed by Redox Dye Mix MB on a plate reader. Phenotype MicroArray assay was purchased from Biolog Inc. (Hayward, CA, USA). A heat map of the resulting data is show in FIG. 6A.

The metabolic substrate utilisation experiments revealed that endothelial cells generated according to the invention preferentially utilise lactose as a substrate. Further experiments were therefore conducted to assess the phenotype and viability of cells cultured with lactose.

H7 hESC-derived endothelial cells and IMR hiPSC-derived endothelial cells generated according to the invention were used for experiments with lactose. Cells were cultured in either EGM2 (control) or EGM2 supplemented with lactose for 1 day, 3 days or 4 days. Lactose (Sigma) was added to glucose-free DMEM medium, supplemented with glutamine (Sigma-Aldrich) prior to being added to the EGM2 media.

High content imaging of the cells was performed, and cell viability was determined by assessing cell number, necrotic marker Topro3, together with mitochondrial membrane potential marker TMRM. The number of CD31+ cells was also determined and compared to EGM2 only controls (FIG. 6B).

Analysis CD31 and FSP expressing cells was also determined and provided an assessment of the endothelial and mesenchymal phenotypes of the cells respectively (FIG. 6B)

### Example 8: Comparison of Genes Expressed by Native Endothelial Cells and Endothelial Cells Differentiated with 2-D and 3-D Protocols

H7 hESC-derived endothelial cells were generated using traditional 2-D methods as described, and in 3-D suspension culture as per example 1.

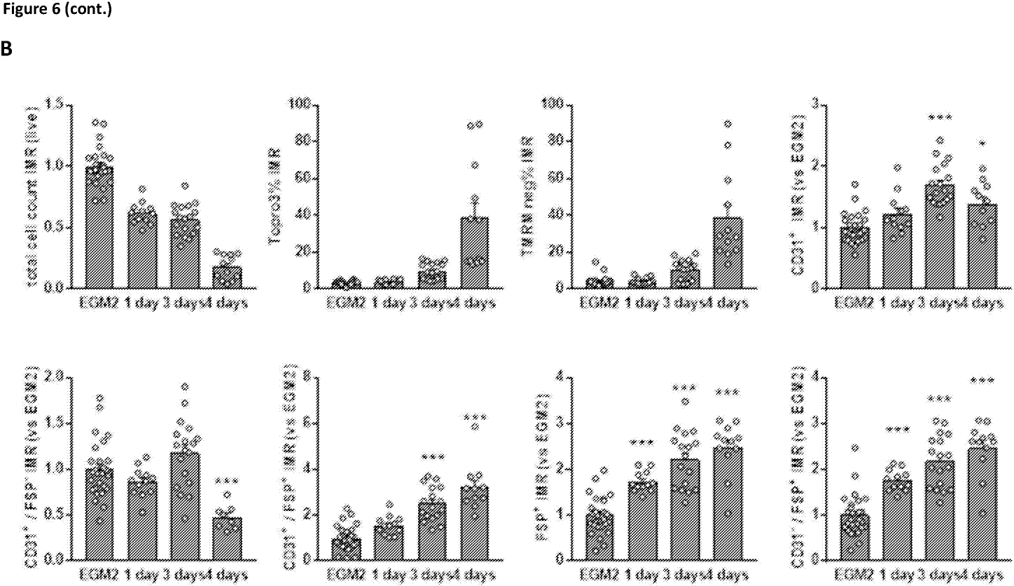

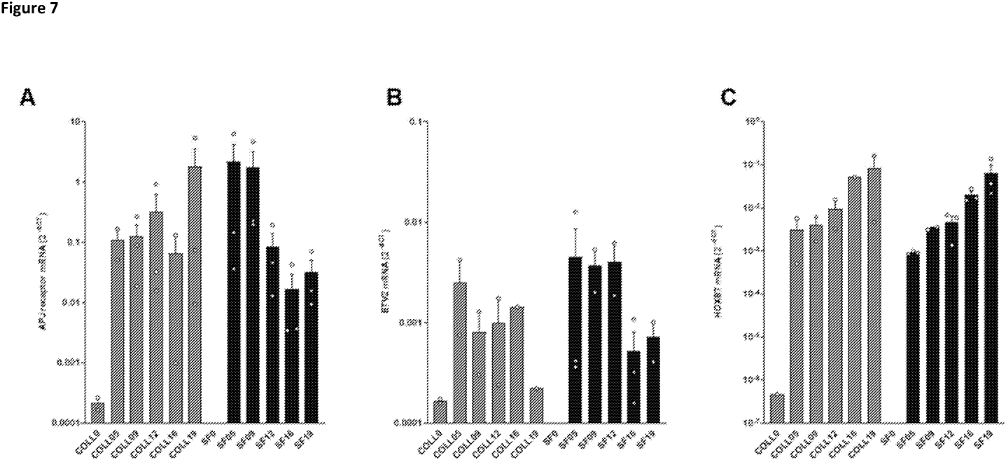

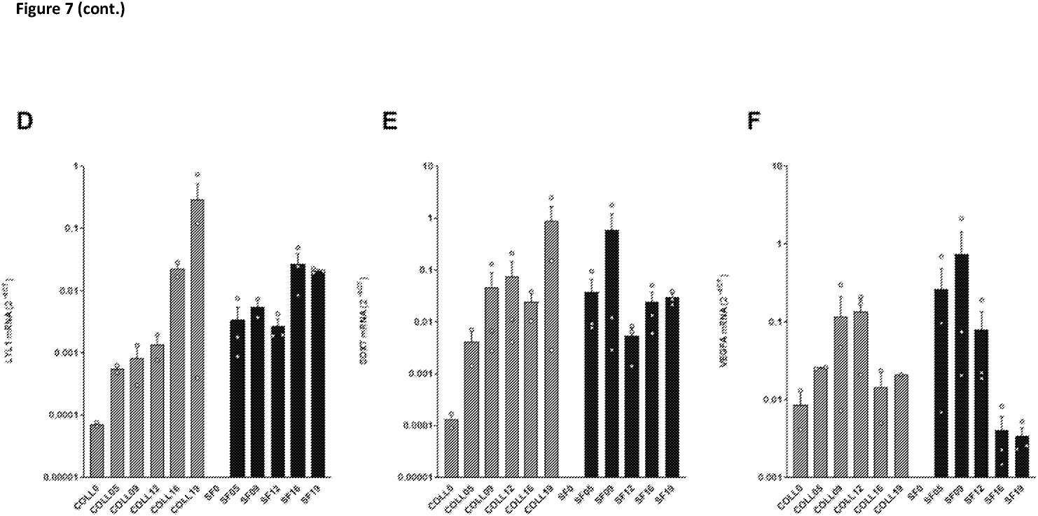

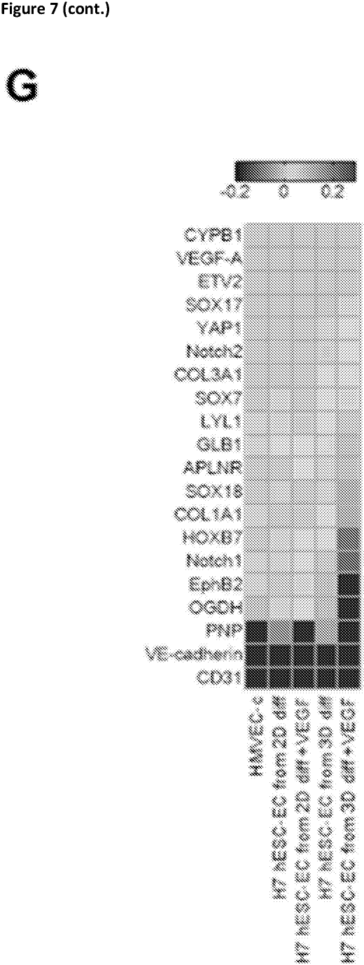

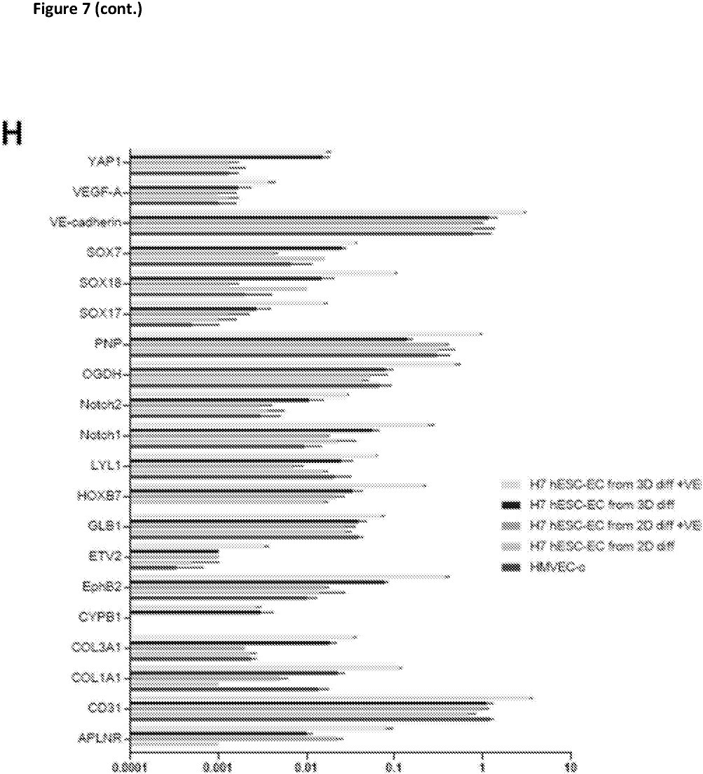

RNA was extracted from both 2-D cultures and 3-D cultures following differentiation (day 0), and 5, 9, 12, 16 and 19 days following differentiation. The expression of APJ, ETV2, HOXB7, LYL1, SOX7 and VEGFA was determined by RT-qPCR as show in FIGS. 7A-F.

The gene expression profile of native endothelial cells (HMVEC-c), hESC-derived endothelial cells generated in 2-D culture with and without VEGF (H7 hESC-EC 2D and H7 hESC-EC 2D+VEGF respectively) and hESC derived endothelial cells generated in 3-D culture with and without VEGF (H7 hESC-EC 3D and H7 hESC-EC 3D+VEGF respectively) was also assessed by RT-qPCR. VEGF was used at a concentration of 500 ng/ml and gene expression assessed at 48-hours. Data from this analysis is shown in the heat map of FIG. 7G and the corresponding bar chart of FIG. 7H. High expression of Notch-1 by the endothelial cells produced by the 3-D culture method of the invention and maintained with high VEGF indicates these endothelial cells have an arterial phenotype.

### Example 9: Proteome Profiling

The proteome profile of native endothelial cells (human aortic smooth muscle cells (hAoSMC); contractile smooth muscle cells (contract SMC); immature smooth muscle cells (immature SMC); human coronary artery endothelial cells (HCAEC); human cardiac microvascular endothelial cells (HMVEC-c)) and hESC-derived endothelial cells generated in 2-D culture and 3-D culture (with and without VEGF) was assessed with a proteome profiler human angiogenesis array kit (R&D System ARY007). Sample preparation and experiment setup followed the product guide. Pixel densities of chemiluminescent signals were analysed by ImageJ software. The results were normalized by a Z score method using the mean and SD of the sample blots.

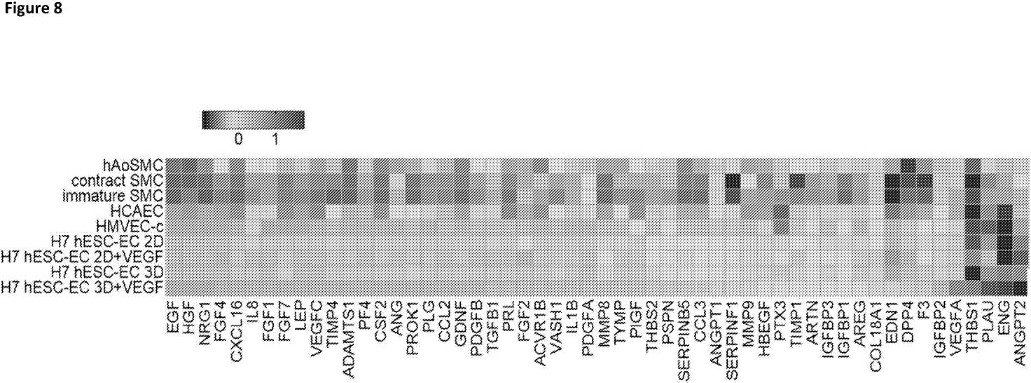

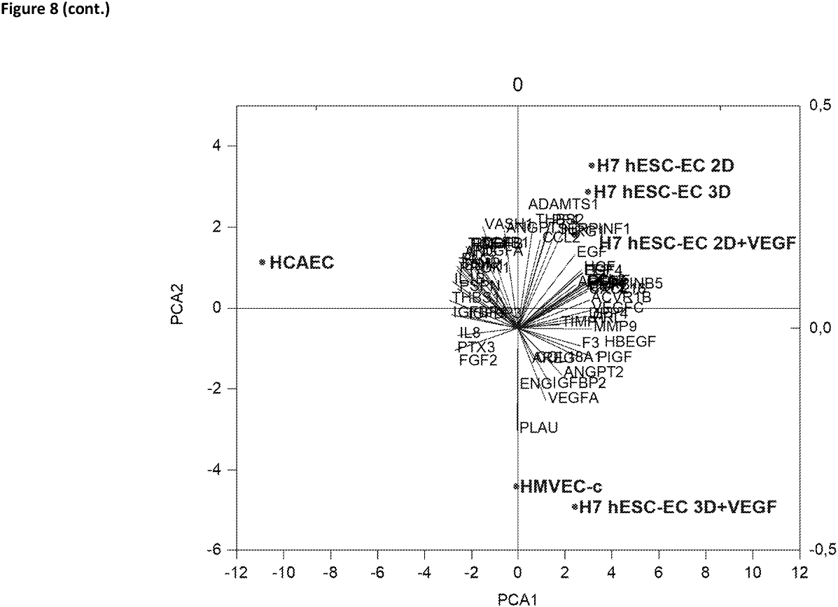

Proteome profiling revealed abundant production of selected proteins in both endothelial cells differentiated in 2-D and 3-D cultures. As show by the heat map in FIG. 8, endothelial cells generated using 3-D culture methods showed a distinct proteome profile endothelial cells generated using 2-D methods.

In more detail a comparison of the four groups of H7 hESC-derived cells was also conducted at the protein level using a Human Angiogenesis Array Kit to determine expression levels of angiogenesis-associated proteins. As ECs derived from different vascular beds have the unique gene and protein expression profiles, two native EC populations: human cardiac microvascular endothelial cell (HMVEC-c) and human coronary artery endothelial cells (HCAEC), representative of the micro-and macro-vasculature beds respectively, were used as comparisons. The two major angiogenic growth factors, VEGF-A and FGF2 were identified to be differentially expressed amongst these cell populations (FIG. 8). Whilst native ECs preferentially express FGF2, H7 derived hESC-ECs did not. Rather, 2D+V and 3D+V H7 hESC-ECs expressed VEGF-A. Conversely, expression of tissue factor (F3), the key activator of coagulation also implicated in angiogenesis, was limited to the groups derived under 3D conditions (3D and 3D+V) indicating that its expression was independent of the beneficial effects of VEGF-A treatment. The angiopoietin (ANGPT) family of growth factors were also screened for. Although expression of ANGPT1 was low across all groups, its pro-angiogenic antagonist, ANGPT2 was expressed in all groups albeit it at low levels in HCAECs. Expression in ‘2D’ and ‘3D’ H7-ECs was in line with HMVEC-c whilst H7-ECs cultured under high VEGF-A (2D+V and 3D+V) had significantly higher ANGPT2 expression with the ‘3D + V’ cells expressing the highest level thus suggesting these cells had an enhanced potential for angiogenesis. Proteins implicated in endothelial dysfunction were also examined. Pentraxin 3 (PTX3), known to impede nitric oxide synthesis and signalling thus exacerbating endothelial dysfunction, was identified to be highly expressed in the native EC populations; however, expression was significantly lower in the non-VEGF groups (2D and 3D) with no expression detected in the cells cultured under high VEGF conditions (2D+V and 3D+V). Thrombospondin 1 (THBS1) is largely perceived to be an inhibitor of angiogenesis. Although present in all groups, HCAECs and ‘3D’ H7 hESC-ECs showed the highest level of expression whilst 2D+V and 3D+V H7 hESC-ECs had the lowest expression. String protein network analysis for ‘3D+V’ H7 hESC-ECs identified VEGF-related networks with VEGF-A acting as the central mediator (FIG. 8B). Overall, principal component analysis subsequently conducted for all six EC populations revealed that ‘3D+V’ H7 hESC-ECs were similar to native HMVEC-c (FIG. 8).

### Example 10: In Vivo Administration of hESC Endothelial Cells Generated in Accordance With The Invention

Endothelial cells generated in accordance with the methods of the present invention were implanted into the heart of an immunocompromised mouse following experimentally induced myocardial infarction. After 1 day, mice were euthanised and the heart dissected, fixed, embedded and sectioned prior to immunohistochemistry using an anti-human CD31 antibody together with Hoechst stain to visualise cell nuclei.

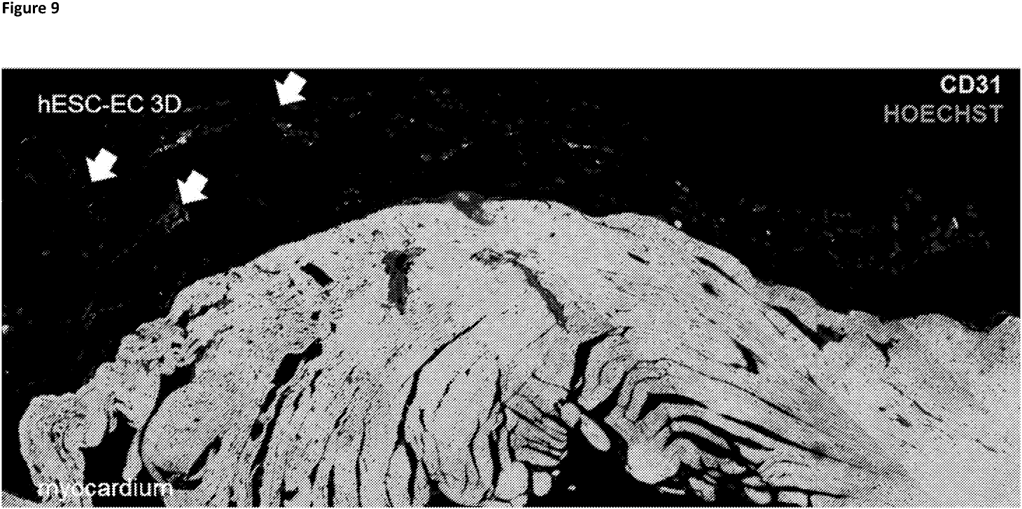

Immunohistochemistry analysis revealed the presence of human CD31+ cells in the epicardial tissue (as shown by the white arrows in FIG. 9 which is a representative image from this experiment).

### Example 11: Differentiation of RC11 hESCs Into Endothelial Cells in 3-D Suspension Culture

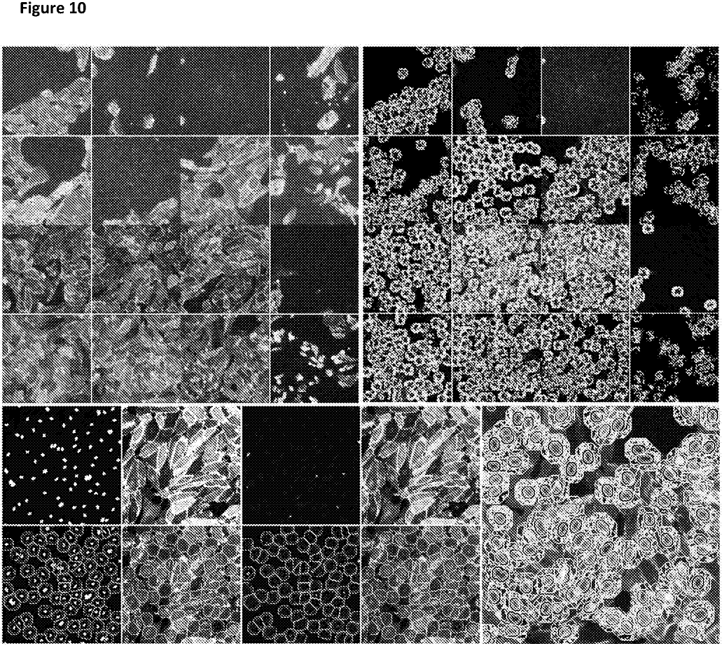

The method of Example 1 was repeated using RC11 hESCs to confirm the general applicability of the 3-D suspension culture method to hESCs hiPSCs. Immunocytochemical analysis of the resulting cells indicated that a homogenous population of endothelial cells was produced from the RC11 cells, with strong CD31 expression (see FIG. 10).

1. A method of generating endothelial cells from pluripotent stem cells, wherein said method comprises:
(a) culturing the pluripotent stem cells in a 3-D suspension culture;
(b) inducing the pluripotent stem cells to undergo endothelial differentiation, wherein inducing comprises:
(i) culturing the pluripotent stem cells for about 24 hours in a first endothelial differentiation medium comprising Activin-A, BMP-4, FGF-2 and VEGF; and
(ii) replacing the first endothelial differentiation medium with a second endothelial differentiation medium comprising BMP-4, FGF-2 and VEGF;
wherein the pluripotent stem cells form cellular aggregates in the suspension.

2. The method of claim 1, wherein said pluripotent stem cells are human pluripotent stem cells, optionally human induced pluripotent stem cells or human embryonic stem cells.

3. The method of claim 1 or 2, wherein said human pluripotent stem cells are H7 cells, IMR 90-4 cells, RC11 cells and/or HUES7 cells, preferably wherein the human pluripotent stem cells are H7 or IMR 90-4 cells.

4. The method of any one of the preceding claims , wherein said endothelial cells are:
(a) CD31⁺ and/or NRP-1^{lo}, preferably CD31⁺ and NRP-^{lo}; and/or
(b) human endothelial cells.

5. The method of any one of the preceding claims , wherein said endothelial cells are KDR⁻ or KDR¹⁰.

6. The method of any one of the preceding claims , wherein said endothelial cells are human endothelial cells which express at least one gene selected from: an F-subgroup Sox transcription factor, preferably SOX7, SOX17 and/or SOX18; LYL1; YAP1; HCLs1; HOXB3; HOXB7; ZNF300; CYP1B1; VEGF-A; VE-Cadherin; PNP; OGDH; NOTCH1; NOTCH2; GLB1; ETV2; Ephrin B2; COL1A1; COL3A1; CD31; APLNR; PLAU; MMP9; ACVR1B; HGF; ERG; Tie2; Angiotensin II; ICAM2; VWF; Fli-1; ALK1; SMAD7; FSP1 and/or SMA-αβ; wherein preferably;
(a) the expression of at least one of SOX7, SOX17, SOX18, LYL1, YAP1, HOXB7, CYP1B1, VEGF-A, VE-Cadherin, PNP, OGDH, NOTCH1, NOTCH2, GLB1, ETV2, Ephrin B2, COL1A1, COL3A1, CD31, and/or APLNR is increased compared with the expression level in endothelial cells generated using traditional 2-D methods and/or native endothelial cells; and/or
(b) the expression level of at least one of HGF and/or FSP1 is decreased compared with the expression level in endothelial cells generated using traditional 2-D methods and/or native endothelial cells.

7. The method of any one of the preceding claims , wherein said human endothelial cells additionally express one or more of SOX7, SOX17, SOX18, LYL1, YAP1, HCLs1, HOXB3, HOXB7, ZNF300, CYP1B1, VEGF-A, VE-Cadherin, PNP, OGDH, NOTCH1, NOTCH2, GLB1, ETV2, Ephrin B2, COL1A1, COL3A1, CD31, APLNR, PLAU, MMP9, ACVR1B, HGF, ERG, Tie2, Angiotensin II, ICAM2, VWF, Fli-1, ALK1, SMAD7, FSP1, SMA-αβ, EGF, NRG1, FGF4, CXCL16, IL8, FGF1, FGF7, LEP, VEGFC, TIMP4, ADAMTS1, PF4, CSF2, ANG, PROK1, PLG, CCL2, GDNF, PDGFB, TGFB1, PRL, FGF2, VASH1, IL1B, PDGFA, MMP8, TYMP, PIGF, THBS2, PSPN, SERPINB5, CCL3, ANGPT1, SERPINF1, HBEGF, PTX3, TIMP1, ARTN, IGFBP3, IGFBP1, AREG, COL18A1, EDN1, DPP4, F3, IGFBP2, THBS1, ENG, ANGPT2, SERPINE1.

8. The method of any one of the preceding claims , wherein:
(a) the concentration of each of Activin-A, BMP-4, FGF-2 and VEGF in the first endothelial differentiation medium is independently selected from a concentration in the range of about 1 ng/ml to 100 ng/ml; wherein preferably the concentration of Activin-A, BMP-4 and FGF-2 is independently selected from a concentration in the range of about 5 to 25 ng/ml and VEGF is present in a concentration in the range of about 5 to 50 ng/ml; more preferably wherein each of Activin-A, BMP-4, FGF-2 and VEGF is present at a concentration of about 10 ng/ml; and/or
(b) the concentration of each of BMP-4, FGF-2 and VEGF in the second endothelial differentiation medium is independently selected from a concentration in the range of 1 ng/ml to 100 ng/ml; wherein preferably the concentration of BMP-4 and FGF-2 is independently selected from a concentration in the range of about 5 to 25 ng/ml and VEGF is present in a concentration in the range of about 0 to 50 ng/ml; more preferably wherein each of BMP-4, FGF-2 and VEGF is present at a concentration of about 10 ng/ml.

9. The method of any one of the preceding claims , wherein said endothelial cells exhibit a stable endothelial cell phenotype.

10. The method of claim 9, wherein said endothelial cells exhibit a stable endothelial cell phenotype for three to 12 passages, preferably five to 10 passages.

11. The method of any one of the preceding claims, wherein said endothelial cells are capable of vascular structure formation in vivo.

12. The method of any one of the preceding claims, wherein said endothelial cells are functional without co-culture, preferably wherein said endothelial cells produce 3-D vascular and/or tubular structures in vitro without co-culture.

13. The method of any one of the preceding claims , wherein said endothelial cells exhibit a microvascular endothelial cell phenotype, preferably a cardiac microvascular endothelial cell phenotype.

14. The method of any one of the preceding claims, wherein the 3-D suspension culture of the pluripotent stem cells is carried out in a stirred-tank bioreactor.

15. The method of claim 14, wherein the stirred-tank bioreactor is operated at between about 20 to 100 rpm, preferably at about 50 rpm.

16. The method of any one of the preceding claims, wherein said culture is:
(a) serum-free;
(b) free of non-human serum albumin;
(c) free from non-human animal derived components; and/or
(d) carried out using human serum albumin as the only animal derived protein in the culture medium.

17. The method of any one of the preceding claims, wherein:
(a) the first endothelial differentiation medium is mTESR1 medium; and
(b) the second endothelial differentiation is StemLine medium.

18. The method of any one of the preceding claims, wherein the culture vessel used for the 3-D suspension culture is feeder-cell free and/or coating-free, preferably both feeder-cell free and coating free.

19. The method of any one of the preceding claims, wherein an adaptive feed rate is used to reduce the concentration of lactate within the 3-D suspension culture.

20. The method of any one of the preceding claims, wherein the pluripotent stem cells are expanded prior to 3-D suspension culture, preferably wherein said expansion comprises culturing the pluripotent stem cells in a pluripotent stem cell expansion medium and passaging the pluripotent stem cells when confluency of at least about 70% is achieved.

21. The method of claim 20, wherein pluripotent stem cell aggregates are retained when transferring the expanded pluripotent stem cells to the 3-D suspension culture.

22. The method of any one of the preceding claims, further comprising the step of isolating one or more endothelial cell, wherein preferably the step of isolating one or more endothelial cell comprises:
(a) FACS, immunoprecipitation or MACS; or
(b) culturing a differentiated cell culture obtained by the method of any one of the preceding claims with a cell culture medium comprising a carbohydrate exclusively metabolised by human endothelial cells.

23. The method of claim 22, wherein the method comprises only one step of isolating one or more endothelial cell.

24. A method of isolating an endothelial cell from a differentiated cell culture, comprising culturing a differentiated cell culture with a cell culture medium comprising a carbohydrate exclusively metabolised by endothelial cells.

25. A method of maintaining an endothelial cell phenotype comprising culturing one or more endothelial cell with a cell culture medium comprising a carbohydrate exclusively metabolised by endothelial cells.

26. The method of any one of claims 22 to 25, wherein the cell culture medium is glucose-free, preferably wherein the carbohydrate is selected from one or more of lactose, meso-tartaric acid, dextrin, maltotriose, D-turanose inosine and/or alpha-keto-glutaric acid.

27. The method of maintaining an endothelial cell phenotype of claim 25 or 26, wherein said endothelial cell phenotype comprises expression of CD31⁺ and NRP-1^{lo}.

28. The method of any one of claims 25 to 27, wherein said endothelial cell phenotype is maintained for three to 12 passages, preferably five to ten passages.

29. The method of isolating an endothelial cell according to any one of claims 22, 23, 24 or 26, or the method of maintain an endothelial cell phenotype according to any one of claims 25 to 28 , wherein the cell culture medium comprises:
(a) VEGF, optionally at a concentration of from about 1 to 100 ng/ml, preferably about 50 ng/ml;
(b) a STAT3 inhibitor; and/or
(c) heparin, optionally at a concentration of from about 1 to 250 mg/ml, preferably about 100 mg/ml.

30. An endothelial cell, preferably a human endothelial cell, obtainable by any one of the methods of claims 1 to 29.

31. A composition comprising an endothelial cell, preferably a human endothelial cell according to claim 30, and optionally a pharmaceutically acceptable excipient.

32. The human endothelial cell according to claim 30, or the composition according to claim 31 for use in the treatment of cardiovascular disease.

33. Use of the human endothelial cell according to claim 30, or the composition according to claim 31, in the manufacture of a medicament for the treatment of vascular disease.
